(19) United States
(12) Patent Application Publication
(10) Pub. No.: US 2009/0136433 A1
(43) Pub. Date: May 28, 2009

(54) Use of Hydrophobin-Polypeptides and Conjugates From Hydrophobin-Polypeptides Having Active and Effect Agents and the Production Thereof and Use Thereof In the Cosmetic Industry

(75) Inventors: Thomas Subkowski, Ladenburg (DE); Marvin Karos, Schwetzingen (DE); Hans-Georg Lemaire, Heidelberg (DE); Heiko Barg, Speyer (DE); Claus Bollschweiler, Limburgerhof (DE)

Correspondence Address: CONNOLLY BOVE LODGE & HUTZ, LLP, P O BOX 2207, WILMINGTON, DE, 19899, US

(73) Assignee: Basf Aktiengesellschaft, Ludwigshafen (DE)

(21) Appl. No.: 11/922,650

(22) PCT Filed: Jun. 23, 2006
(86) PCT No.: PCT/EP2006/063485

§ 371 Date: Dec. 20, 2007

(30) Foreign Application Priority Data
Jun. 24, 2005 (DE) 10 2005 029 704.8

(51) Int. Cl. A61K 8/64; A61K 8/66; A61Q 17/04; A61K 38/16; A01N 37/18; A01P 3/00; A01P 7/04

(52) U.S. Cl. 424/59; 514/12; 424/94.1; 424/70.14

(57) ABSTRACT

Cosmetic composition for the treatment of keratin-containing materials, mucosa and teeth, comprising at least one hydrophobin polypeptide sequence (i)

(I)

Xₙ-C¹-X₁₋₅₀-C²-X₀₋₅-C³-Xₚ-C⁴-X₁₋₁₀₀-C⁵-X₁₋₅₀-C⁶-
X₀₋₅-C⁷-X₁₋₅₀-C⁸-Xₘ

### PRIOR ART

Hydrophobins are small proteins of about 100 amino acids which are characteristic of filamentous fungi and do not occur in other organisms. Recently, hydrophobin-like proteins have been discovered in Streptomyces coelicolor, which are referred to as “chaplins” and likewise have highly surface-active properties. At water/air interfaces chaplins are able to assemble to form amyloid-like fibrils (Classen et al. 2003 Genes Dev 1714-1726; Elliot et al. 2003, Genes Dev. 17, 1727-1740).

Hydrophobins are distributed in a water-insoluble form on the surface of various fungal structures, such as, for example, aerial hyphae, spores, and fruiting bodies. The genes for hydrophobins were isolated from ascomycetes, deuteromycetes and basidiomycetes. Some fungi comprise more than one hydrophobin gene, e.g. Schizophyllum commune, Coprinus cinereus, Aspergillus nidulans. Various hydrophobins are of course involved in different stages of fungal development. The hydrophobins are here presumably responsible for various functions (van Wetter et al., 2000, Mol. Microbiol., 36, 201-210; Kershaw et al. 1998, Fungal Genet. Biol, 1998, 23, 18-33).

Biological functions for hydrophobins which have been described are not only the reduction in the surface tension of water to generate aerial hyphae, but also the hydrophobicization of spores (Wösten et al. 1999, Curr. Biol., 19, 1985-88; Bell et al. 1992, Genes Dev., 6, 2382-2394). In addition, hydrophobins serve to line gas channels in fruiting bodies of lichen and as components in the recognition system of plant surfaces by fungal pathogens (Lugones et al. 1999, Mycol. Res., 103, 635-640; Hamer & Talbot 1998, Curr. Opinion Microbiol., Volume 1, 693-697).

Complementation experiments have shown that hydrophobins are able to functionally replace themselves within one class to a certain degree.

The hydrophobins known to date can only be prepared in moderate yield and purity using customary protein chemistry purification and isolation methods. Attempts using genetic engineering techniques to provide relatively large amounts of hydrophobins have also hitherto been unsuccessful.

US 20030217419A1 describes the use of the hydrophobin SC3 from Schizophyllumg commune for cosmetic preparations.

### OBJECT

It was an object of the present invention to provide novel polypeptides which have a high affinity to keratin or keratin-containing substances such as skin, nails or hair and/or to mucosa and/or teeth. Such polypeptides are suitable for the cosmetic and pharmaceutical treatment of keratin-containing structures, in particular of hair, nails and skin, or of mucosa or teeth, and as an anchor for a large number of active substances and effect substances.

It was also an object of the present invention to provide novel keratin-binding effector molecules which have a binding peptide with high affinity to keratin or keratin-containing substances such as skin or hair, and to which certain effector molecules are bound. Such keratin-binding effector molecules permit a high local concentration of effector molecules on the keratin, so-called “targeting” or a long action time on the keratin.

### DESCRIPTION OF THE INVENTION

The invention provides cosmetic compositions for the treatment of keratin-containing materials, mucosa and teeth, comprising at least one hydrophobin polypeptide sequence (i) of the general structural formula (I) in a cosmetically compatible medium.

Structural formula (I)

(I)

Xₙ-C¹-X₁₋₅₀-C²-X₀₋₅-C³-Xₚ-C⁴-X₁₋₁₀₀-C⁵-X₁₋₅₀-C⁶-
X₀₋₅-C⁷-X₁₋₅₀-C⁸-Xₘ

where X can be any of the 20 naturally occurring amino acids (Phe, Leu, Ser, Tyr, Cys, Trp, Pro, His, Gin, Arg, Ile, Met, Thr, Asn, Lys, Val, Ala, Asp, Glu, Gly) and the indices alongside X are the number of amino acids, where the indices n and m are numbers between 0 and 500, preferably between 15 and 300, p is a number between 1 and 250, preferably 1-100, and C is cysteine, alanine, serine, glycine, methionine or threonine, where at least four of the radicals named as C are cysteine, with the proviso that at least one of the peptide sequences abbreviated to Xₙ or Xₘ or Xₚ is a peptide sequence which is at least 20 amino acids in length, which is naturally not linked to a hydrophobin,
which, following coating of a glass surface, bring about a change in the contact angle of at least 20°.

The amino acids named as C¹ to C⁸ are preferably cysteines; they can, however, also be replaced by other amino acids of similar spatial arrangement, preferably by alanine, serine, threonine, methionine or glycine. However, at least four, preferably at least 5, particularly preferably at least 6 and in particular at least 7, of the positions C¹ to C⁸ should consist of cysteines. Cysteines may be present in the proteins according to the invention either in reduced form, or form disulfide bridges with one another. Particular preference is given to the intramolecular formation of C—C bridges, in particular those with at least one, preferably 2, particularly preferably 3 and very particularly preferably 4, intramolecular disulfide bridges. In the case of the above-described replacement of cysteines by amino acids of similar spatial arrangement, such C positions are advantageously exchanged in pairs which can form intramolecular disulfide bridges with one another.

If cysteines, serines, alanines, glycines, methionines or threonines are also used in the positions referred to as X, the numbering of the individual C positions in the general formulae can change accordingly.

Particularly advantageous polypeptides (i) are those of the general formula (II)

(II)

Xₙ-C¹-X₃₋₂₅-C²-X₀₋₂-C³-X₅₋₅₀-C⁴-X₂₋₃₅-C⁵-X₂₋₁₅-
C⁶-X₀₋₂-C⁷-X₃₋₃₅-C⁸-Xₘ

where X is any of the 20 naturally occurring amino acids (Phe, Leu, Ser, Tyr, Cys, Trp, Pro, His, Gln, Arg, Ile, Met, Thr, Asn, Lys, Val, Ala, Asp, Glu, Gly) and the indices alongside X are the number of amino acids, where the indices n and m are numbers between 2 and 300 and C is cysteine, alanine, serine, glycine, methionine or threonine, where at least four of the radicals named as C are cysteine, with the proviso that at least one of the peptide sequences abbreviated to Xₙ or Xₘ is a polypeptide sequence which is at least 35 amino acids in length, which is naturally not linked to a hydrophobin,
which, following coating of a glass surface, bring about a change in the contact angle of at least 20°.

Of very particular advantage are those polypeptides (i) of the general formula (III)

(III)

Xₙ-C¹-X₅₋₉-C²-C³-X₁₁₋₃₉-C⁴-X₂₋₂₃-C⁵-X₅₋₉-C⁶-C⁷-
X₆₋₁₆-C⁸-Xₘ

where X may be any of the 20 naturally occurring amino acids (Phe, Leu, Ser, Tyr, Cys, Trp, Pro, His, Gln, Arg, Ile, Met, Thr, Asn, Lys, Val, Ala, Asp, Glu, Gly) and the indices alongside X are the number of amino acids, where the indices n and m are numbers between 0 and 200 and C is cysteine, alanine, serine, glycine, methionine or threonine, where at least six of the radicals named as C are cysteine, with the proviso that at least one of the peptide sequences abbreviated to Xₙ or Xₘ is a peptide sequence which is at least 40 amino acids in length, which is naturally not linked to a hydrophobin, which, following coating of a glass surface, bring about a change in the contact angle of at least 20°.

Preferred embodiments of the described invention are polypeptides with the general structural formula (I) (II) or (III), where this structural formula comprises at least one hydrophobin of class I, preferably at least one hydrophobin dewA, rodA, hypA, hypB, sr3, basf1, basf2, or parts or derivatives thereof. The specified hydrophobins are characterized structurally in the sequence protocol below. It is also possible for a plurality of hydrophobins, preferably 2 or 3, of identical or different structure to be linked together or to be linked to a corresponding suitable polypeptide sequence, which is naturally not bonded to a hydrophobin.

Particularly preferred embodiments of the present invention are the novel proteins with the polypeptide sequences depicted in SEQ ID NO: 20, 22, 24, and the nucleic acid sequences which encode for these, in particular the sequences according to SEQ ID NO: 19, 21, 23. Proteins which arise starting from the polypeptide sequences depicted in SEQ ID NO. 22, 22 or 24 as a result of exchange, insertion or deletion of at least one, up to 10, preferably 5, particularly preferably 5%, of all amino acids and which still have at least 50% of the biological property of the starting proteins are also particularly preferred embodiments. Biological property of the proteins is understood here as meaning the change in the contact angle as described in Example 10.

The proteins according to the invention carry at least one position, which is abbreviated to Xₙ or Xₘ or Xₚ, a polypeptide sequence of at least 20, preferably at least 35, particularly preferably at least 50 and in particular at least 100, amino acids (also referred to below as fusion partners), which is not naturally linked to a hydrophobin. This expression is intended to mean that the proteins according to the invention consist of a hydrophobin part and a fusion partner part which do not occur together in this form in nature.

The fusion partner part can be chosen from a large number of proteins. It is also possible for a plurality of fusion partners to be linked to a hydrophobin part, for example on the amino terminus (Xₙ) and on the carboxy terminus (Xₘ) of the hydrophobin part. However, it is also possible, for example, for two fusion partners to be linked to one position (Xₙ or Xₘ) of the protein according to the invention.

Particularly preferred fusion partners are those polypeptide sequences which lead to the protein according to the invention being able to coat glass surfaces, and the glass surface treated with protein being resistant to a detergent treatment, as is described in detail (e.g. 1% SDS/80° C./10 min) in the experimental section (Example 10).

Particularly suitable fusion partners are polypeptides which occur naturally in microorganisms, in particular in E. coli or Bacillus subtilis. Examples of such fusion partners are the sequences yaad (SEQ ID NO:15 and 16), yaae (SEQ ID NO:17 and 18), and thioredoxin. Fragments or derivatives of these specified sequences which comprise only part, preferably 10-90%, particularly preferably 25-75%, of said sequences are also highly suitable. A deletion on the C terminal end is preferred here; for example a yaad fragment which consists only of the first 75 N-terminal amino acids, or in which individual amino acids, or nucleotides have been altered compared with the specified sequence. For example, at the C-terminal end of the sequences yaad and yaae, additional amino acids, in particular two additional amino acids, preferably the amino acids Arg, Ser, can also be attached. Furthermore, additional amino acids compared with the naturally occurring sequence, for example the amino acid No. 2 (Gly) in SEQ ID NO: 17 and 18, may preferably be inserted into the sequence yaae.

Further preferred fusion partners are keratin-binding domains, for example those which occur in human desmoplacin or which can be derived from this through customary genetic engineering methods such as amino acid desubstitution, insertion or deletion.

The binding to keratin can easily be checked using the experiments described in the experimental section.

In addition, at the linkage sites of two fusion partners, additional amino acids may also be inserted which arise as a result of recognition sites for restriction endonucleases either being newly created or deactivated at the nucleic acid level.

The proteins according to the invention can also be modified in their polypeptide sequence, for example by glycosylation, acetylation or else through chemical crosslinking, for example with glutardialdehyde.

One property of the proteins according to the invention is the change in surface properties if the surfaces are coated with the proteins. The change in the surface properties can be determined experimentally by measuring the contact angle of a drop of water before and after coating the surface with the protein according to the invention and calculating the difference between the two measurements.

The precise experimental conditions for measuring the contact angle are laid down in the experimental section in Example 10. Under these conditions, the proteins according to the invention have the property to increase the contact angle by at least 20, preferably 25, particularly preferably 30 degrees.

In the hydrophobin part of the hydrophobins known to date the positions of the polar and nonpolar amino acids are preserved, which is evident from a characteristic hydrophobicity plot. Differences in the biophysical properties and in the hydrophobicity led to the classification of the hydrophobins known to date into two classes, I and II (Wessels et al. 1994, Ann. Rev. Phytopathol., 32, 413-437).

The assembled membranes from hydrophobin class I are to a large extent insoluble (even to 1% SDS at elevated temperature) and can only be dissociated again using concentrated trifluoroacetic acid (TFA) or formic acid. In contrast to this, the assembled forms of class II hydrophobins are less stable. They can be dissolved again using just 60% strength ethanol, or 1% SDS (at room temperature). This high stability to solvents and detergents is a particular property of the hydrophobins and differentiates coatings with the polypeptides according to the invention from “non-specific” protein coatings which a large number of proteins form on surfaces.

A comparison of the amino acid sequences shows that the length of the region between cysteine C³ and C⁴ in the case of class II hydrophobins is significantly shorter than in the case of class I hydrophobins.

Class II hydrophobins also have more charged amino acids than class I.

The invention further provides hydrophobin-comprising effector molecules consisting of

(i) at least one hydrophobin,
(ii) one effector molecule which is naturally not linked to the hydrophobin (i).

The hydrophobin (i) can be represented by all known hydrophobin polypeptides. Particularly suitable hydrophobins (i) are the abovementioned polypeptides (i) of the general structural formula (I), (II) or (III).

Preferred hydrophobin polypeptide sequences (i) comprise an amino acid sequence according to SEQ ID NO: 1-24.

Likewise included according to the invention are “functional equivalents” of the specifically disclosed hydrophobin polypeptide sequences (i) and the use thereof in the methods according to the invention.

“Functional equivalents” or analogs of the specifically disclosed polypeptides (i) are, for the purposes of the present invention, polypeptides which differ therefrom and which additionally have the desired biological activity such as, for example, keratin binding. Thus, for example, “functional equivalents” are understood as meaning hydrophobin polypeptide sequences which, according to one of the binding tests described in Examples 12/13, show a binding of at least 10%, preferably at least 50%, particularly preferably 75%, very particularly preferably 90%, of the binding shown by a hydrophobin of SEQ ID NO: 1-24 in the binding tests according to Examples 12/13.

Examples of suitable amino acid substitutions are given in the table below:

| Original radical | Substitution examples |
|---|---|
| Ala | Ser |
| Arg | Lys |
| Asn | Gln; His |
| Asp | Glu |
| Cys | Ser |
| Gln | Asn |
| Glu | Asp |
| Gly | Pro |
| His | Asn; Gln |
| Ile | Leu; Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

According to the invention, “functional equivalents” are understood as meaning in particular also muteins which have, in at least one sequence position of the abovementioned amino acid sequences, an amino acid other than that specifically mentioned, but nevertheless have one of the abovementioned biological activities. “Functional equivalents” thus comprise the muteins obtainable by one or more amino acid additions, substitutions, deletions and/or inversions, it being possible for said modifications to occur in any sequence position as long as they lead to a mutein having the property profile according to the invention.

“Functional equivalents” in the above sense are also “precursors” of the described polypeptides, and “functional derivatives” and “salts” of the polypeptides.

“Precursors” are here natural or synthetic precursors of the polypeptides with or without the desired biological activity.

The term “salts” is understood as meaning both salts of carboxyl groups and acid addition salts of amino groups of the protein molecules according to the invention. Salts of carboxyl groups can be prepared in a manner known per se and comprise inorganic salts, such as, for example, sodium, calcium, ammonium, iron and zinc salts, and salts with organic bases, such as, for example, amines, such as triethanolamine, arginine, lysine, piperidine and the like. Acid addition salts, such as, for example, salts with mineral acids, such as hydrochloric acid or sulfuric acid and salts with organic acids such as acetic acid and oxalic acid are likewise provided by the invention.

“Functional derivatives” of polypeptides according to the invention can likewise be prepared on functional amino acid side groups or on the N- or C-terminal end thereof by means of known techniques. Such derivatives comprise, for example, aliphatic esters of carboxylic acid groups, amides of carboxylic acid groups, obtainable by reaction with ammonia or with a primary or secondary amine; N-acyl derivatives of free amino groups prepared by reaction with acyl groups; or O-acyl derivatives of free hydroxy groups prepared by reaction with acyl groups.

“Functional equivalents” naturally also comprise polypeptides which are obtainable from other organisms, and naturally occurring variants. It is possible for example to establish ranges of homologous sequence regions by comparison of sequences, and to ascertain equivalent enzymes based on the specific requirements of the invention.

“Functional equivalents” likewise comprise fragments, preferably single domains or sequence motifs, of the polypeptides according to the invention, which have, for example, the desired biological function.

“Functional equivalents” are also fusion proteins which comprise one of the abovementioned hydrophobin polypeptide sequences or functional equivalents derived therefrom and at least one further, heterologous sequence which is functionally different therefrom and is in functional N- or C-terminal linkage (i.e. without significant mutual functional impairment of the parts of the fusion protein). Nonlimiting examples of such heterologous sequences are, for example, signal peptides or enzymes.

“Functional equivalents” also included according to the invention are homologs of the specifically disclosed proteins. These have at least 50%, preferably at least 75%, in particular at least 85%, such as, for example, 90%, 95% or 99%, homology with one of the specifically disclosed amino acid sequences calculated by the algorithm of Pearson and Lipman, Proc. Natl. Acad. Sci. (USA) 85(8), 1988, 2444-2448. A percentage homology of a homologous polypeptide according to the invention means in particular percentage identity of the amino acid residues based on the total length of one of the amino acid sequences specifically described herein.

In the case of possible protein glycosylation, “functional equivalents” according to the invention comprise proteins of the type described above in deglycosylated or glycosylated form, and modified forms obtainable by altering the glycosylation pattern.

Homologs of the polypeptides (i) according to the invention can be generated by mutagenesis, e.g. by point mutation or truncation of the protein.

Homologs of the polypeptides according to the invention can be identified by screening combinatorial libraries of mutants, such as, for example, truncation mutants. For example, a library of protein variants can be generated by combinatorial mutagenesis at the nucleic acid level, such as, for example, by enzymatic ligation of a mixture of synthetic oligonucleotides. There is a large number of methods which can be used to prepare libraries of potential homologs from a degenerate oligonucleotide sequence. Chemical synthesis of a degenerate gene sequence can be carried out in an automatic DNA synthesizer, and the synthetic gene can then be ligated into a suitable expression vector. The use of a degenerate set of genes makes it possible to provide all of the sequences which encode the desired set of potential protein sequences in one mixture. Methods for synthesizing degenerate oligonucleotides are known to the person skilled in the art (e.g. Narang, S. A. (1983) Tetrahedron 39:3; Itakura et al. (1984) Annu. Rev. Biochem. 53:323; Itakura et al., (1984) Science 198:1056; Ike et al. (1983) Nucleic Acids Res. 11:477).

Several techniques are known in the prior art for screening gene products in combinatorial libraries which have been prepared by point mutations or truncation, and for screening cDNA libraries for gene products having a selected property. These techniques can be adapted to the rapid screening of gene libraries which have been generated by combinatorial mutagenesis of homologs according to the invention. The most commonly used techniques for screening large gene libraries, which are subject to high-throughput analysis, comprise the cloning of the gene library in replicable expression vectors, transformation of suitable cells with the resulting vector library and expression of the combinatorial genes under conditions under which detection of the desired activity facilitates isolation of the vector which encodes the gene whose product has been detected. Recursive ensemble mutagenesis (REM), a technique which increases the frequency of functional mutants in the libraries, can be used in combination with the screening tests to identify homologs (Arkin and Yourvan (1992) PNAS 89:7811-7815; Delgrave et al. (1993) Protein Engineering 6(3):327-331).

A particularly advantageous embodiment of the invention are hydrophobin-comprising effector molecules in which the hydrophobin is represented by a polypeptide sequence (i) which comprises at least one of the following hydrophobin polypeptide sequences,
a) polypeptide sequence of the general structural formula (I),
b) polypeptide sequence which is modified in up to 60%, preferably up to 75%, particularly preferably up to 90%, of the amino acid positions compared to (a),
with the proviso that the keratin-binding of the hydrophobin polypeptide sequence (b) is at least 10% of the value which the hydrophobin polypeptide sequence (a) has measured in the test according to Examples 12 and 13.

Modifying amino acids means here amino acid substitutions, insertions and deletions or any combinations of these three possibilities.

Preference is given to using hydrophobin polypeptide sequences (i) which have a highly specific affinity for the desired organism. For uses in skin cosmetics, consequently, preference is given to using hydrophobin polypeptide sequences (i) which have a particularly high affinity to human skin keratin. For uses in hair cosmetics, preference is given to those hydrophobin polypeptide sequences which have a particularly high affinity to human hair keratin.

For uses in the pet sector, preference is accordingly given to those hydrophobin polypeptide sequences (i) which have a particularly high affinity to the corresponding keratin, for example dog keratin or cat keratin.

It is, however, also possible to use more than one hydrophobin polypeptide sequence (i) in the effector molecule according to the invention. A plurality of copies of the same hydrophobin polypeptide sequence (i) can also be connected in series in order, for example, to achieve higher binding.

The hydrophobins according to the invention with keratin-binding properties have a broad field of use in human cosmetics, in particular skin care and hair care, and animal care.

The hydrophobin polypeptides (i) according to the invention are preferably used for hair and skin cosmetics. They permit a high concentration and long action time of skin care or skin-protecting effector substances.

### Effector Molecules (ii)

In the text below effector molecules (ii) are understood as meaning molecules which have a certain foreseeable effect. These may either be protein-like molecules, such as enzymes, or non-proteinogenic molecules, such as dyes, photoprotective agents, vitamins and fatty acids, or compounds comprising metal ions.

Among the protein-like effector molecules, enzymes and antibodies are preferred. Among the enzymes, the following are preferred as effector molecules (ii): oxidases, peroxidases, proteases, tyrosinases, metal-binding enzymes, lactoperoxidase, lysozyme, amyloglycosidase, glucose oxidase, superoxide dismutase, photolyase, calalase.

Highly suitable protein-like effector molecules (ii) are also hydrolysates of proteins from vegetable and animal sources, for example hydrolysates of proteins of marine origin or silk hydrolysates.

Among the non-protein-like effector molecules (ii), preference is given to dyes, for example semipermanent dyes or oxidation dyes. In the case of the reactive dyes, one component is preferably linked as effector molecule (ii) to the keratin-binding hydrophobin polypeptide sequence (i) and then oxidatively coupled to the second dye component at the site of action, i.e. after binding to the hair.

Suitable dyes are all customary hair dyes for the molecules according to the invention. Suitable dyes are known to the person skilled in the art from cosmetics handbooks, for example Schrader, Grundlagen und Rezepturen der Kosmetika [Fundamentals and Formulations of Cosmetics], Hüthig Verlag, Heidelberg, 1989, ISBN 3-7785-1491-1.

Further suitable effector molecules (ii) are carotenoids. According to the invention, carotenoids are understood as meaning the following compounds: β-carotene, lycopene, lutein, astaxanthin, zeaxanthin, cryptoxanthin, citranaxanthin, canthaxanthin, bixin, β-apo-4-carotenal, β-apo-8-carotenal, β-apo-8-carotenic esters, individually or as a mixture. Preferably used carotenoids are β-carotene, lycopene, lutein, astaxanthin, zeaxanthin, citranaxanthin and canthaxanthin.

For the purposes of the present invention, retinoids means vitamin A alcohol (retinol) and its derivatives, such as vitamin A aldehyde (retinal), vitamin A acid (retinoic acid) and vitamin A esters (e.g. retinyl acetate, retinyl propionate and retinyl palmitate). The term retinoic acid comprises here both all-trans retinoic acid and also 13-cis retinoic acid. The terms retinol and retinal preferably comprise the all-trans compounds. A preferred retinoid used for the suspensions according to the invention is all-trans retinol, referred to below as retinol.

Further preferred effector molecules (ii) are vitamins, in particular vitamin A and esters thereof.

Vitamins, provitamins and vitamin precursors from the groups A, C, E end F, in particular 3,4-didehydroretinol, β-carotene (provitamin of vitamin A), ascorbic acid (vitamin C), and the palmitic esters, glucosides or phosphates of ascorbic acid, tocopherols, in particular α-tocopherol and its esters, e.g. the acetate, the nicotinate, the phosphate and the succinate; also vitamin F, which is understood as meaning essential fatty acids, in particular linoleic acid, linolenic acid and arachidonic acid;

Vitamin A and its derivatives and provitamins advantageously exhibit a particular skin-smoothing effect.

The vitamins, provitamins or vitamin precursors of the vitamin B group or derivatives thereof, and the derivatives of 2-furanone to be used with preference according to the invention include, inter alia:

Vitamin B₁, trivial name thiamine, chemical name 3-[(4′-amino-2′-methyl-5′-pyrimidinyl) methyl]-5-(2-hydroxyethyl)-4-methylthiazolium chloride.

Vitamin B₂, trivial name riboflavin, chemical name 7,8-dimethyl-10-(1-D-ribityl)-benzo[g]pteridine-2,4(3H, 10H)-dione. Riboflavin occurs in free form e.g. in whey, other riboflavin derivatives can be isolated from bacteria and yeasts. One stereoisomer of riboflavin which is likewise suitable according to the invention is lyxoflavin, which can be isolated from fish meal or liver and carries a D-arabityl radical instead of the D-ribityl.

Vitamin B₃. Listed under this name are often the compounds nicotinic acid and nicotinamide (niacinamide). According to the invention, preference is given to nicotinamide.

Vitamin B₅ (pantothenic acid and panthenol). Preference is given to using panthenol. Derivatives of panthenol which can be used according to the invention are, in particular, the esters and ethers of panthenol, and cationically derivatized panthenols. In a further preferred embodiment of the invention, derivatives of 2-furanone can also be used in addition to pantothenic acid or pathenol. Particularly preferred derivatives are the substances, also commercially available, dihydro-3-hydroxy-4,4-dimethyl-2(3H)-furanone with trivial name pantolactone (Merck), 4-hydroxymethyl-γ-butyrolactone (Merck), 3,3-dimethyl-2-hydroxy-γ-butyrolactone (Aldrich) and 2,5-dihydro-5-methoxy-2-furanone (Merck), where all stereoisomers are expressly included.

These compounds advantageously bestow the keratin-binding effector molecules according to the invention with moisturizing and skin-calming properties.

Vitamin B₆, which is understood not as being a uniform substance, but the derivatives of 5-hydroxymethyl-2-methylpyridin-3-ol known under the trivial names pyridoxine, pyridoxamine and pyridoxal.

Vitamin B₇ (biotin), which is also referred to as vitamin H or “skin vitamin”. Biotin is (3aS,4S,6aR)-2-oxohexahydrothienol[3,4-d]imidazole-4-valeric acid.

Panthenol, pantolactone, nicotinamide and biotin are very particularly preferred according to the invention.

According to the invention, suitable derivatives (salts, esters, sugars, nucleotides, nucleosides, peptides and lipids) can. As lipophilic, oil-soluble antioxidants from this group, preference is given to tocopherol and derivatives thereof, gallic esters, flavonoids and carotenoids, and butylhydroxytoluene/anisole. As water-soluble antioxidants, preference is given to amino acids, e.g. tyrosine and cysteine and derivatives thereof, and tannins, in particular those of vegetable origin.

Triterpenes, in particular triterpenoic acids, such as ursolic acid, rosemarinic acid, betulinic acid, boswellic acid and bryonolic acid.

Further preferred effector molecules (ii) are UV photoprotective filters. These are understood as meaning organic substances which are able to absorb ultraviolet rays and give off the absorbed energy again in the form of longer-wave radiation, e.g. heat. The organic substances may be oil-soluble or water-soluble.

Examples of oil-soluble UV-B filters which may be used are the following substances:

3-benzylidenecamphor and derivatives thereof, e.g. 3-(4-methylbenzylidene)camphor;
4-aminobenzoic acid derivatives, preferably 2-ethylhexyl 4-(dimethylamino)benzoate, 2-octyl 4-(dimethylamino)benzoate and amyl 4-(dimethylamino)benzoate;
esters of cinnamic acid, preferably 2-ethylhexyl 4-methoxycinnamate, propyl 4-methoxycinnamate, isoamyl 4-methoxycinnamate, isopentyl 4-methoxycinnamate, 2-ethylhexyl 2-cyano-3-phenylcinnamate (octocrylene);
esters of salicylic acid, preferably 2-ethylhexyl salicylate, 4-isopropylbenzyl salicylate, homomethyl salicylate;
derivatives of benzophenbne, preferably 2-hydroxy-4-methoxybenzophenone, 2-hydroxy-4-methoxy-4′-methylbenzophenone, 2,2′-dihydroxy-4-methoxybenzophenone;
esters of benzalmalonic acid, preferably di-2-ethylhexyl 4-methoxybenzmalonate;
triazine derivatives, such as, for example, 2,4,6-trianilino(p-carbo-2′-ethyl-1′-hexyloxy)-1,3,5-triazine (octyltriazone) and dioctylbutamidotriazone (Uvasorb® HEB):
propane-1,3-diones, such as, for example, 1-(4-tert-butylphenyl)-3-(4′-methoxyphenyl)propane-1,3-dione.

Suitable water-soluble substances are:

2-phenylbenzimidazole-5-sulfonic acid and alkali metal, alkaline earth metal, ammonium, alkylammonium, alkanolammonium and glucammonium salts thereof;
sulfonic acid derivatives of benzophenones, preferably 2-hydroxy-4-methoxybenzo-phenone-5-sulfonic acid and its salts;
sulfonic acid derivatives of 3-benzylidenecamphor, such as, for example, 4-(2-oxo-3-bornylidenemethyl)benzenesulfonic acid and 2-methyl-5-(2-oxo-3-bornylidene)sulfonic acid and salts thereof.

Particular preference is given to the use of esters of cinnamic acid, preferably 2-ethylhexyl 4-methoxycinnamate, isopentyl 4-methoxycinnamate, 2-ethylhexyl 2-cyano-3-phenylcinnamate (octocylene).

Furthermore, the use of derivatives of benzophenone, in particular 2-hydroxy-4-methoxybenzophenone, 2-hydroxy-4-methoxy-4′-methylbenzophenone, 2,2′-dihydroxy-4-methoxybenzophenone, and the use of propane-1,3-diones, such as, for example, 1-(4-tert-butylphenyl)-3-(4′-methoxyphenyl)propane-1,3-dione, is preferred.

Suitable typical UV-A filters are:

derivatives of benzoylmethane, such as, for example, 1-(4′-tert-butylphenyl)-3-(4′-methoxyphenyl)propane-1,3-dione, 4-tert-butyl-4′-methoxydibenzoylmethane or 1-phenyl-3-(4′-isopropylphenyl)propane-1,3-dione;
aminohydroxy-substituted derivatives of benzophenones, such as, for example, N,N-diethylaminohydroxybenzoyl n-hexylbenzoate.

The UV-A and UV-B filters can of course also be used in mixtures.

Further photoprotective filters which may be used are, however, also other insoluble pigments, e.g. finely disperse metal oxides and salts, such as, for example, titanium dioxide, iron oxide, aluminum oxide, cerium oxide, zirconium oxide, silicates (talc), barium sulfate and zinc stearate. The particles should have an average diameter of less than 100 nm, preferably between 5 and 50 nm and in particular between 15 and 30 nm.

Besides the two abovementioned groups of primary photoprotective substances, it is also possible to use secondary photoprotective agents of the antioxidant type, which interrupt the photochemical reaction chain which is triggered when UV radiation penetrates into the skin. Typical examples thereof are superoxide dismutase, catalase, tocopherols (vitamin E) and ascorbic acid (vitamin C).

A further group are antiirritatives which have an antiinflammatory effect on skin damaged by UV light. Such substances are, for example, bisabolol, phytol and phytantriol.

Linkage of the effector molecules (ii) to the keratin-binding hydrophobin-hydrophobin polypeptide sequence (i)

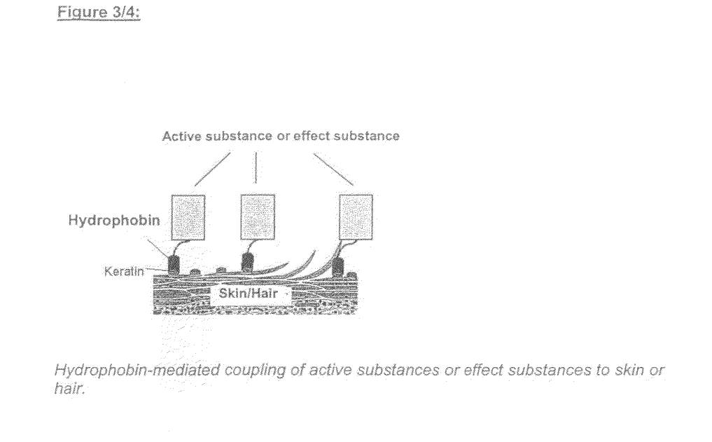

The effector molecules (ii) are bonded to a hydrophobin polypeptide sequence (i) which has a binding affinity to a keratin. The bond between (i) and (ii) can either be covalent or based on ionic or van-der Waals interactions (FIG. 3).

Preference is given to a covalent linkage. This can take place, for example, via the side chains of the hydrophobin polypeptide sequence (i), in particular via amino functions or carboxylate functions or thiol functions. Preference is given to a linkage via the amino functions of one or more lysine radicals, one or more thiol groups of cysteine radicals or via the N-terminal or C-terminal function of the hydrophobin polypeptide (i). The linkage of the effector molecules (ii) to the hydrophobin polypeptide sequence (i) can either be direct, i.e. as covalent linkage of two chemical functions already present in (i) and (ii), for example an amino function of (i) is linked to a carboxylate function of (ii) to give the acid amide. The linkage can, however, also take place via a so-called linker, i.e. an at least bifunctional molecule which, with one function, enters into a bond with (i), and with another function is linked to (ii).

If the effector molecule (ii) likewise consists of a polypeptide sequence, the linkage of (i) and (ii) can take place through a so-called fusion protein, i.e. a common polypeptide sequence which consists of the two partial sequences (i) and (ii).

Between (i) and (ii) it is also possible for so-called spacer elements to be incorporated, for example polypeptide sequences which have a potential cleavage site for a protease, lipase, esterase, phosphatase, hydrolase, or polypeptide sequences which permit easy purification of the fusion protein, for example so-called His tags, i.e. oligohistidine radicals.

The linkage in the case of a non-protein-like effector molecule with the hydrophobin polypeptide sequence (i) preferably takes place through functionalizable radicals (side groups) on the hydrophobin polypeptide (i) which enter into a covalent bond with a chemical function of the effector molecule.

Preference is given here to the binding linkage via an amino, thiol or hydroxy function of the hydrophobin polypeptide (i) which, for example, can enter into a corresponding amide, thioester or ester bond with a carboxyl function of the effector molecule (ii), if appropriate following activation.

A further preferred linkage of the hydrophobin polypeptide sequence (i) with an effector molecule (ii) is the use of a tailored linker. Such a linker has two or more so-called anchor groups with which it can link the hydrophobin polypeptide sequence (i) and one or more effector molecules (ii). For example, an anchor group for (i) may be a thiol function, by means of which the linker can enter into a disulfide bond with a cysteine radical of the polypeptide (i). An anchor group of (ii) can, for example, be a carboxyl function by means of which the linker can enter into an ester bond with a hydroxyl function of the effector molecule (ii).

The use of such tailored linkers permits the precise adaptation of the linkage to the desired effector molecule. Moreover, it is thereby possible to link two or more effector molecules with a hydrophobin polypeptide sequence (i) in a defined manner.

The linker used is governed by the functionality to be coupled. Of suitability are, for example, molecules which couple to polypeptides (i) by means of sulfhydryl-reactive groups, e.g. maleimides, pydridyldisulfides, α-haloacetyls, vinylsulfone and to effector molecules (ii) by means of
sulfhydryl-reactive groups, e.g. maleimides, pydridyldisulfides, α-haloacetyls, vinylsulfones) amine-reactive groups (e.g. succinimidyl esters, carbodiimides, hydroxymethylphosphine, imido esters, PFP esters etc.)
sugars and oxidized sugar-reactive groups (e.g. hydrazides etc.)
carboxy-reactive groups (e.g. carbodiimides etc.)
hydroxyl-reactive groups (e.g. isocyanates etc.)
thymine-reactive groups (e.g. psoralen etc.)
unselective groups (e.g. aryl azides etc.)
photoactivatable groups (e.g. perfluorophenyl azide etc.)
metal-complexing groups (e.g. EDTA, hexahis, ferritin)
antibodies and antibody fragments (e.g. single-chain antibodies, F(ab) fragments of antibodies, catalytic antibodies).

Alternatively, a direct coupling between active substance/effect substance and the keratin-binding domains can be carried out, for example by means of carbodiimides, glutardialdehyde or other crosslinkers known to the person skilled in the art.

The linker can be stable, thermocleavable, photocleavable or enzymatically cleavable (particularly by lipases, esterases, proteases, phosphatases, hydrolases etc.). Corresponding chemical structures are known to the person skilled in the art and are integrated between the molecule parts (i) and (ii).

Examples of enzymatically cleavable linkers which can be used with the molecules according to the invention are specified, for example, in WO 98/01406, the entire contents of which are hereby expressly incorporated by reference.

The keratin-binding hydrophobin effector molecules according to the invention have a broad field of application in human cosmetics, in particular skin care and hair care, animal care.

Preferably, the keratin-binding hydrophobin effector molecules according to the invention are used for skin cosmetics, nail cosmetics and hair cosmetics. They permit a high concentration and long action time of skin care, nail care and hair care or skin-protecting, nail-protecting and hair-protecting effector substances.

Suitable auxiliaries and additives for the preparation of hair cosmetic or skin cosmetic preparations are known to a person skilled in the art and can be found in cosmetics handbooks, for example Schrader, Grundlagen und Rezepturen der Kosmetika [Fundamentals and Formulations of Cosmetics], Hüthig Verlag, Heidelberg, 1989, ISBN 3-7785-1491-1.

The cosmetic compositions according to the invention may be skin cosmetic, hair cosmetic, dermatological, hygiene or pharmaceutical compositions.

Preferably, the compositions according to the invention are in the form of a gel, foam, spray, ointment, cream, emulsion, suspension, lotion, milk or paste. If desired, liposomes or microspheres may also be used.

The cosmetically or pharmaceutically active compositions according to the invention can additionally comprise cosmetically and/or dermatologically active substances and auxiliaries.

Preferably, the cosmetic compositions according to the invention comprise at least one hydrophobin polypeptide sequence (i) as defined above, and at least one constituent different therefrom which is chosen from cosmetically active substances, emulsifiers, surfactants, preservatives, perfume oils, thickeners, hair polymers, hair and skin conditioners, graft polymers, water-soluble or dispersible silicone-containing polymers, photoprotective agents, bleaches, gel formers, care agents, colorants, tinting agents, tanning agents, dyes, pigments, bodying agents, humectants, refatting agents, collagen, protein hydrolysates, lipids, antioxidants, antifoams, antistats, emollients and softeners.

Customary thickeners in such formulations are crosslinked polyacrylic acids and derivatives thereof, polysaccharides and derivatives thereof, such as xanthan gum, agar agar, alginates or tyloses, cellulose derivatives, e.g. carboxymethylcellulose or hydroxycarboxymethylcellulose, fatty alcohols, monoglycerides and fatty acids, polyvinyl alcohol and polyvinylpyrrolidone. Preference is given to using nonionic thickeners.

Suitable cosmetically and/or dermatologically active substances are, for example, coloring active substances, skin and hair pigmentation agents, tinting agents, tanning agents, bleaches, keratin-hardening substances, antimicrobial active substances, photofilter active substances, repellent active substances, hyperemic substances, substances with a keratolytic and keratoplastic effect, antidandruff active substances, antiphlogistics, substances with a keratinizing effect, antioxidative active substances or active substances which act as free-radical scavengers, skin moisturizing or humectant substances, refatting active substances, antierythematous or antiallergic active substances and mixtures thereof.

Active substances which tan the skin artificially and which are suitable for tanning the skin without natural or artificial irradiation with UV rays are, for example, dihydroxyacetone, alloxan and walnut shell extract. Suitable keratin-hardening substances are usually active substances as are also used in antiperspirants, such as, for example, potassium aluminum sulfate, aluminum hydroxychloride, aluminum lactate, etc.

Antimicrobial active substances are used to destroy microorganisms or to inhibit their growth and thus serve both as preservatives and also as deodorizing substance which reduces the formation or the intensity of body odor. These include, for example, customary preservatives known to the person skilled in the art, such as p-hydroxybenzoic esters, imidazolidinylurea, formaldehyde, sorbic acid, benzoic acid, salicylic acid, etc. Such deodorizing substances are, for example, zinc ricinoleate, triclosan, undecylenic alkylolamides, triethyl citrate, chlorhexidine etc.

Suitable photofilter active substances are substances which absorb UV rays in the UV-B and/or UV-A region. Suitable UV filters are, for example, 2,4,6-triaryl-1,3,5-triazines in which the aryl groups may in each case carry at least one substituent which is preferably chosen from hydroxy, alkoxy, specifically methoxy, alkoxycarbonyl, specifically methoxycarbonyl and ethoxycarbonyl and mixtures thereof. Also suitable are p-aminobenzoic esters, cinnamic esters, benzophenones, camphor derivatives, and pigments which stop UV rays, such as titanium dioxide, talc and zinc oxide.

Suitable repellent active substances are compounds which are able to drive away or keep certain animals, in particular insects, away from humans. These include, for example, 2-ethyl-1,3-hexanediol, N,N-diethyl-m-toluamide etc. Suitable substances with hyperemic activity, which stimulate the flow of blood through the skin, are, for example essential oils, such as dwarf pine extract, lavender extract, rosemary extract, juniper berry extract, roast chestnut extract, birch leaf extract, hayflower extract, ethyl acetate, camphor, menthol, peppermint oil, rosemary extract, eucalyptus oil, etc. Suitable keratolytic and keratoplastic substances are, for example, salicylic acid, calcium thioglycolate, thioglycolic acid and its salts, sulfur etc. Suitable antidandruff active substances are, for example, sulfur, sulfur polyethylene glycol sorbitan monooleate, sulfur ricinol polyethoxylate, zinc pyrithione, aluminum pyrithione, etc. Suitable antiphlogistics, which counter skin irritations, are, for example, allantoin, bisabolol, dragosantol, camomile extract, panthenol, etc.

The cosmetic compositions according to the invention can comprise, as cosmetic and/or pharmaceutical active substance (and also if appropriate as auxiliary), at least one cosmetically or pharmaceutically acceptable polymer which differs from the polymers which form the polyelectrolyte complex used according to the invention. These include, quite generally, cationic, amphoteric and neutral polymers.

Suitable polymers are, for example, cationic polymers with the INCl name Poly-quaternium, e.g. copolymers of vinylpyrrolidone/N-vinylimidazolium salts (Luviquat FC, Luviquat HM, Luviquat MS, Luviquat&commat, Care), copolymers of N-vinylpyrrolidone/dimethylaminoethyl methacrylate, quaternized with diethyl sulfate (Luviquat PQ 11), copolymers of N-vinylcaprolactam/N-vinylpyrrolidone/N-vinylimidazolium salts (Luviquat E Hold), cationic cellulose derivatives (Polyquaternium-4 and -10), acrylamido copolymers (Polyquaternium-7) and chitosan.

Suitable cationic (quaternized) polymers are also Merquat (polymer based on dimethyldiallylammonium chloride), Gafquat (quaternary polymers which are produced by the reaction of polyvinylpyrrolidone with quaternary ammonium compounds), Polymer JR (hydroxyethylcellulose with cationic groups) and plant-based cationic polymers, e.g. guar polymers such as the Jaguar grades from Rhodia.

Further suitable polymers are also neutral polymers, such as polyvinylpyrrolidones, copolymers of N-vinylpyrrolidone and vinyl acetate and/or vinyl propionate, polysiloxanes, polyvinylcaprolactam and other copolymers with N-vinylpyrrolidone, polyethyleneimines and salts thereof, polyvinylamines and salts thereof, cellulose derivatives, polyaspartic acid salts and derivatives. These include, for example, Luviflex 0 Swing (partially saponified copolymer of polyvinyl acetate and polyethylene glycol, BASF).

Suitable polymers are also nonionic, water-soluble or water-dispersible polymers or oligomers, such as polyvinylcaprolactam, e.g. Luviskol 0 Plus (BASF), or polyvinylpyrrolidone and copolymers thereof, in particular with vinyl esters, such as vinyl acetate, e.g. Luviskol 0 VA 37 (BASF), polyamides, e.g. based on itaconic acid and aliphatic diamines, as are described, for example, in DE-A-43 33 238.

Suitable polymers are also amphoteric or zwitterionic polymers, such as the octylacrylamide/methyl methacrylate/tert-butylaminoethyl methacrylate/hydroxypropyl methacrylate copolymers obtainable under the names Amphomer (National Starch), and zwitterionic polymers as are disclosed, for example, in the German patent applications DE39 29 973, DE 21 50 557, DE28 17 369 and DE 3708 451. Acrylamidopropyltrimethylammonium chloride/acrylic acid or methacrylic acid copolymers and alkali metal and ammonium salts thereof are preferred zwitterionic polymers. Further suitable zwitterionic polymers are methacroylethylbetaine/methacrylate copolymers, which are available commercially under the name Amersette (AMERCHOL), and copolymers of hydroxyethyl methacrylate, methyl methacrylate, N,N-dimethylaminoethyl methacrylate and acrylic acid (Jordapon (D)).

Suitable polymers are also nonionic, siloxane-containing, water-soluble or water-dispersible polymers, e.g. polyether siloxanes, such as Tegopren 0 (Goldschmidt) or Besi&commat (Wacker).

The formulation base of pharmaceutical compositions according to the invention preferably comprises pharmaceutically acceptable auxiliaries. Pharmaceutically acceptable auxiliaries are those which are known for use in the field of pharmacy, food technology and related fields, in particular those listed in the relevant pharmacopoeia (e.g. DAB Ph. Eur. BP NF) and other auxiliaries whose properties do not preclude a physiological application.

Suitable auxiliaries may be: lubricants, wetting agents, emulsifying and suspending agents, preserving agents, antioxidants, antiirritatives, chelating agents, emulsion stabilizers, film formers, gel formers, odor-masking agents, resins, hydrocolloids, solvents, solubility promoters, neutralizing agents, permeation accelerators, pigments, quaternary ammonium compounds, refatting and superfatting agents-, ointment-, cream- or oil-based substances, silicone derivatives, stabilizers, sterilizers, propellants, drying agents, opacifiers, thickeners, waxes, softeners, white oil. Formulation in this regard is based on specialist knowledge, as given, for example, in Fiedler, H. P. Lexikon der Hilfsstoffe für Pharmazie, Kosmetik und angrenzende Gebiete [Lexicon of Auxiliaries for Pharmacy, Cosmetics and related fields], 4th ed., Aulendorf: ECV-Editio-Kantor-Verlag, 1996.

To prepare the dermatological compositions according to the invention, the active substances can be mixed or diluted with a suitable auxiliary (excipient). Excipients may be solid, semi solid or liquid materials which can serve as a vehicle, carrier or medium for the active substance. Further auxiliaries are added, if desired, in the manner known to the person skilled in the art. In addition, the polymers and dispersions are suitable as auxiliaries in pharmacy, preferably as or in coating(s) or binder(s) for solid drug forms. They can also be used in creams and as tablet coatings and tablet binders.

According to a preferred embodiment, the compositions according to the invention are a skin-cleansing composition.

Preferred skin-cleansing compositions are soaps of liquid to gel-like consistency, such as transparent soaps, luxury soaps, deodorant soaps, cream soaps, baby soaps, skin protection soaps, abrasive soaps and syndets, pasty soaps, soft soaps and washing pastes, liquid washing, shower and bath preparations, such as washing lotions, shower baths and gels, foam baths, oil baths and scrub preparations, shaving foams, lotions and creams.

According to a further preferred embodiment, the compositions according to the invention are cosmetic compositions for the care and protection of the skin, nail care compositions or preparations for decorative cosmetics.

Suitable skin cosmetic compositions are, for example, face tonics, face masks, deodorants and other cosmetic lotions. Compositions for use in decorative cosmetics comprise, for example, concealing sticks, stage makeup, mascara and eye shadows, lipsticks, kohl pencils, eyeliners, blushers, powders and eyebrow pencils.

Furthermore, the hydrophobin polypeptide sequences (i) can be used in nose strips for pore cleansing, in antiacne compositions, repellents, shaving compositions, hair-removal compositions, intimate care compositions, foot care compositions, and in babycare.

The skincare compositions according to the invention are, in particular, W/O or O/W skin creams, day and night creams, eye creams, face creams, antiwrinkle creams, moisturizing creams, bleach creams, vitamin creams, skin lotions, care lotions and moisturizing lotions.

Skin cosmetic and dermatological compositions based on the above-described poly-electrolyte complexes exhibit advantageous effects. The polymers can, inter alia, contribute to the moisturization and conditioning of the skin and to an improvement in the feel of the skin. The polymers can also act as thickeners in the formulations. By adding the polymers according to the invention, in certain formulations a considerable improvement in the skin compatibility can be achieved.

Skin cosmetic and dermatological compositions comprise preferably at least one hydrophobin polypeptide sequence (i) in an amount of from about 0.000001 to 10% by weight, preferably 0.0001 to 1% by weight, based on the total weight of the composition.

Particularly photoprotective compositions based on the hydrophobin polypeptide sequences (i) have the property of increasing the residence time of the UV-absorbing ingredients compared to customary auxiliaries such as polyvinylpyrrolidone.

Depending on the field of use, the compositions according to the invention can be applied in a form suitable for skin care, such as, for example, as a cream, foam, gel, stick, mousse, milk, spray (pump spray or propellant-containing spray) or lotion.

Besides the hydrophobin polypeptide sequences (i) and suitable carriers, the skin cosmetic preparations can also comprise further active substances and auxiliaries customary in skin cosmetics, as described above. These include preferably emulsifiers, preservatives, perfume oils, cosmetic active substances, such as phytantriol, vitamin A, E and C, retinol, bisabolol, panthenol, photoprotective agents, bleaches, colorants, tints, tanning agents, collagen, protein hydrolysates, stabilizers, pH regulators, dyes, salts, thickeners, gel formers, consistency regulators, silicones, moisturizers, refatting agents and further customary additives.

Preferred oil and fat components of the skin cosmetic and dermatological compositions are the abovementioned mineral and synthetic oils, such as, for example, paraffins, silicone oils and aliphatic hydrocarbons having more than 8 carbon atoms, animal and vegetable oils, such as, for example, sunflower oil, coconut oil, avocado oil, olive oil, lanolin, or waxes, fatty acids, fatty acid esters, such as, for example, triglycerides of the C6-C30-fatty acids, wax esters, such as, for example, jojoba oil, fatty alcohols, vaseline, hydrogenated lanolin and acetylated lanolin, and mixtures thereof.

The hydrophobin polypeptide sequences (i) according to the invention can also be mixed with conventional polymers if specific properties are to be established.

To establish certain properties, such as, for example, improvement in the feel to the touch, the spreading behavior, the water resistance and/or the binding of active substances and auxiliaries, such as pigments, the skin cosmetic and dermatological preparations can additionally also comprise conditioning substances based on silicone compounds.

Suitable silicone compounds are, for example, polyalkylsiloxanes, polyarylsiloxanes, polyarylalkylsiloxanes, polyethersiloxanes or silicone resins.

The cosmetic or dermatological preparations are prepared by customary methods known to the person skilled in the art.

Preferably, the cosmetic and dermatological compositions are in the form of emulsions, in particular water-in-oil (W/O) or oil-in-water (O/W) emulsions.

It is, however, also possible to choose other types of formulations, for example hydrodispersions, gels, oils, oleogels, multiple emulsions, for example in the form of W/O/W or O/W/O emulsions, anhydrous ointments or ointment bases, etc.

The emulsions are prepared by known methods. Besides at least one hydrophobin polypeptide sequence (i), the emulsions generally comprise customary constituents, such as fatty alcohols, fatty acid esters and, in particular, fatty acid triglycerides, fatty acids, lanolin and derivatives thereof, natural or synthetic oils or waxes and emulsifiers in the presence of water. The selection of the additives specific to the type of emulsion and the preparation of suitable emulsions is described, for example, in Schrader, Grundlagen und Rezepturen der Kosmetika [Fundamentals and Formulations of Cosmetics], Hüthig Buch Verlag, Heidelberg, 2nd edition, 1989, third part, which is hereby expressly incorporated by reference.

A suitable emulsion, e.g. for a skin cream etc., generally comprises an aqueous phase which is emulsified by means of a suitable emulsifier system in an oil or fat phase. To provide the aqueous phase, a polyelectrolyte complex can be used.

Preferred fat components which may be present in the fatty phase of the emulsions are: hydrocarbon oils, such as paraffin oil, purcellin oil, perhydrosqualene and solutions of microcrystalline waxes in these oils; animal or vegetable oils, such as sweet almond oil, avocado oil, calophylum oil, lanolin and derivatives thereof, castor oil, sesame oil, olive oil, jojoba oil, karité oil, hoplostethus oil, mineral oils whose distillation start point under atmospheric pressure is about 250° C. and whose distillation end point is 410° C., such as, for example, vaseline oil, esters of saturated or unsaturated fatty acids, such as alkyl myristates, e.g. isopropyl, butyl or cetyl myristate, hexadecyl stearate, ethyl or isopropyl palmitate, octanoic or decanoic acid triglycerides and cetyl ricinoleate.

The fatty phase can also comprise silicone oils soluble in other oils, such as dimethylpolysiloxane, methylphenylpolysiloxane and the silicone glycol copolymer, fatty acids and fatty alcohols.

Besides the hydrophobin polypeptide sequences (i) it is also possible to use waxes, such as, for example, carnauba wax, candelilla wax, beeswax, microcrystalline wax, ozokerite wax and Ca, Mg and Al oleates, myristates, linoleates and stearates.

In addition, an emulsion according to the invention can be in the form of an O/W emulsion. Such an emulsion usually comprises an oil phase, emulsifiers which stabilize the oil phase in the water phase, and an aqueous phase, which is usually present in thickened form. Suitable emulsifiers are preferably O/W emulsifiers, such as polyglycerol esters, sorbitan esters or partially esterified glycerides.

According to a further preferred embodiment, the compositions according to the invention are a shower gel, a shampoo formulation or a bath preparation.

Such formulations comprise at least one hydrophobin polypeptide sequence (i) and customary anionic surfactants as base surfactants and amphoteric and/or nonionic surfactants as cosurfactants. Further suitable active substances and/or auxiliaries are generally chosen from lipids, perfume oils, dyes, organic acids, preservatives and antioxidants, and thickeners/gel formers, skin conditioning agents and moisturizers.

These formulations comprise preferably 2 to 50% by weight, preferably 5 to 40% by weight, particularly preferably 8 to 30% by weight, of surfactants, based on the total weight of the formulation.

In the washing, shower and bath preparations it is possible to use all anionic, neutral, amphoteric or cationic surfactants customarily used in body-cleansing compositions.

Suitable anionic surfactants are, for example, alkyl sulfates, alkyl ether sulfates, alkyl-sulfonates, alkylarylsulfonates, alkyl succinates, alkyl sulfosuccinates, N-alkoyl-sarcosinates, acyl taurates, acyl isothionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units, in the molecule. These include, for example, sodium lauryl sulfate, ammonium lauryl sulfate, sodium lauryl ether sulfate, ammonium lauryl ether sulfate, sodium lauryl sarcosinate, sodium oleyl succinate, ammonium lauryl sulfosuccinate, sodium dodecylbenzenesulfonate, triethanolamine dodecylbenzenesulfonate.

Suitable amphoteric surfactants are, for example, alkylbetaines, alkylamidopropylbetaines, alkylsulfobetaines, alkyl glycinates, alkyl carboxyglycinates, alkyl amphoacetates or amphopropionates, alkyl amphodiacetates or amphodipropionates.

For example, cocodimethylsulfopropylbetaine, laurylbetaine, cocamidopropylbetaine or sodium cocamphopropionate can be used.

Suitable nonionic surfactants are, for example, the reaction products of aliphatic alcohols or alkylphenols having 6 to 20 carbon atoms in the alkyl chain, which may be linear or branched, with ethylene oxide and/or propylene oxide. The amount of alkylene oxide is about 6 to 60 moles per mole of alcohol. In addition, alkylamine oxides, mono- or dialkylalkanolamides, fatty acid esters of polyethylene glycols, ethoxylated fatty acid amides, alkyl polyglycosides or sorbitan ether esters are suitable.

Furthermore, the washing, shower and bath preparations can comprise customary cationic surfactants, such as, for example, quaternary ammonium compounds, for example cetyltrimethylammonium chloride.

In addition, the shower gel/shampoo formulations can comprise thickeners, such as, for example, sodium chloride, PEG-55, propylene glycol oleate, PEG-120 methylglucose dioleate and others, and preservatives, further active substances and auxiliaries and water.

According to a further preferred embodiment, the compositions according to the invention are a hair-treatment composition.

Hair-treatment compositions according to the invention preferably comprise at least one hydrophobin polypeptide sequence (i) in an amount in the range from about 0.000001 to 10% by weight, preferably 0.00001 to 1% by weight, based on the total weight of the composition.

Preferably, the hair-treatment compositions according to the invention are in the form of a setting foam, hair mousse, hair gel, shampoo, hair spray, hair foam, ends fluid, neutralizers for permanent waves, hair colorants and bleaches or hot-oil treatments. Depending on the field of use, the hair cosmetic preparations can be applied as (aerosol) spray, (aerosol) foam, gel, gel spray, cream, lotion or wax. Hair sprays here comprise both aerosol sprays and also pump sprays without propellant gas. Hair foams comprise both aerosol foams and also pump foams without propellant gas. Hair sprays and hair foams comprise preferably predominantly or exclusively water-soluble or water-dispersible components. If the compounds used in the hair sprays and hair foams according to the invention are water-dispersible, they can be applied in the form of aqueous microdispersions with particle diameters of from usually 1 to 350 nm, preferably 1 to 250 nm. The solids contents of these preparations here are usually in a range from about 0.5 to 20% by weight. These microdispersions generally require no emulsifiers or surfactants for their stabilization.

The hair cosmetic formulations according to the invention comprise, in a preferred embodiment, a) 0.000001 to 10% by weight of at least one hydrophobin polypeptide sequence (i), b) 20 to 99.95% by weight of water and/or alcohol, c) 0 to 50% by weight of at least one propellant gas, d) 0 to 5% by weight of at least one emulsifier, e) 0 to 3% by weight of at least one thickener, and up to 25% by weight of further constituents.

Alcohol is understood as meaning all alcohols customary in cosmetics, e.g. ethanol, isopropanol, n-propanol.

Further constituents are understood as meaning the additives customary in cosmetics, for example propellants, antifoams, inferface-active compounds, i.e. surfactants, emulsifiers, foam formers and solubilizers. The interface-active compounds used may be anionic, cationic, amphoteric or neutral. Further customary constituents may also be, for example, preservatives, perfume oils, opacifiers, active substances, UV filters, care substances, such as panthenol, collagen, vitamins, protein hydrolysates, alpha- and beta-hydroxycarboxylic acids, stabilizers, pH regulators, dyes, viscosity regulators, gel formers, salts, moisturizers, refatting agents, complexing agents and further customary additives.

These also include all styling and conditioner polymers known in cosmetics which can be used in combination with the hydrophobin polypeptide sequences (i) according to the invention if very specific properties are to be established.

Suitable conventional hair cosmetic polymers are, for example, the above-mentioned cationic, anionic, neutral, nonionic and amphoteric polymers, which are hereby incorporated by reference.

To establish certain properties, the preparations can additionally also comprise conditioning substances based on silicone compounds. Suitable silicone compounds are, for example, polyalkylsiloxanes, polyarylsiloxanes, polyarylalkylsiloxanes, polyethersiloxanes, silicone resins or dimethicone copolyols (CTFA) and aminofunctional silicone compounds, such as amodimethicones (CTFA).

The polymers according to the invention are particularly suitable as setting agents in hair styling preparations, in particular hair sprays (aerosol sprays and pump sprays without propellant gas) and hair foams (aerosol foams and pump foams without propellant gas).

In a preferred embodiment, spray preparations comprise a) 0.000001 to 10% by weight of at least one hydrophobin polypeptide sequence (i), b) 90 to 99.9% by weight of water and/or alcohol, c) 0 to 70% by weight of at least one propellant, d) 0 to 20% by weight of further constituents.

Propellants are the propellants customarily used for hair sprays or aerosol foams. Preference is given to mixtures of propane/butane, pentane, dimethyl ether, 1,1-difluoroethane (HFC-152 a), carbon dioxide, nitrogen or compressed air.

A formulation for aerosol hair foams preferred according to the invention comprises a) 0.000001 to 10% by weight of at least one hydrophobin polypeptide sequence (i), b) 90 to 99.9% by weight of water and/or alcohol, c) 5 to 20% by weight of a propellant, d) 0.1 to 5% by weight of an emulsifier, e) 0 to 10% by weight of further constituents.

Emulsifiers which can be used are all of the emulsifiers customarily used in hair foams. Suitable emulsifiers may be nonionic, cationic or anionic or amphoteric.

Examples of nonionic emulsifiers (INCl nomenclature) are laureths, e.g. laureth-4; ceteths, e.g. cetheth-1, polyethylene glycol cetyl ethers, ceteareths, e.g. cetheareth-25, polyglycol fatty acid glycerides, hydroxylated lecithin, lactyl esters of fatty acids, alkyl polyglycosides.

Examples of cationic emulsifiers are cetyldimethyl-2-hydroxyethylammonium dihydrogenphosphate, cetyltrimonium chloride, cetyltrimonium bromide, cocotrimonium methyl sulfate, quaternium-1 to x (INCl).

Anionic emulsifiers can, for example, be chosen from the group of alkyl sulfates, alkyl ether sulfates, alkylsulfonates, alkylarylsulfonates, alkyl succinates, alkyl sulfosuccinates, N-alkoylsarcosinates, acyl taurates, acyl isethionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units, in the molecule.

A preparation suitable according to the invention for styling gels can, for example, have the following composition: a) 0.000001 to 10% by weight of at least one hydrophobin polypeptide sequence (i), b) 80 to 99.85% by weight of water and/or alcohol, c) 0 to 3% by weight, preferably 0.05 to 2% by weight, of a gel former, d) 0 to 20% by weight of further constituents.

In general, the hydrophobin polypeptide sequences (i) used according to the invention already have a “self-thickening” effect, meaning that in many cases the use of gel formers can be dispensed with when preparing gels. Their use may, however, be advantageous in order to establish specific rheological or other application properties of the gels. Gel formers which may be used are all gel formers customary in cosmetics. These include slightly crosslinked polyacrylic acid, for example carbomer (INCl), cellulose derivatives, e.g. hydroxypropylcellulose, hydroxyethylcellulose, catonically modified celluloses, polysaccharides, e.g. xanthan gum, caprylic/capric triglyceride, sodium acrylate copolymers, polyquaternium-32 (and) Paraffinum Liquidum (INCl), sodium acrylate copolymers (and) Paraffinum Liquidum (and) PPG-1 trideceth-6, acrylamidopropyltrimonium chloride/acrylamide copolymers, steareth-10 allyl ether, acrylate copolymers, polyquaternium-37 (and) Paraffinum Liquidum (and) PPG-1 trideceth-6, polyquaternium 37 (and) propylene glycol dicaprate dicaprylate (and) PPG-1 trideceth-6, polyquaternium-7, polyquaternium-44.

The hydrophobin polypeptide sequences (i) according to the invention can be used as conditioners in cosmetic preparations.

A preparation comprising the hydrophobin polypeptide sequences (i) according to the invention can preferably be used in shampoo formulations as setting agent and/or conditioner. Preferred shampoo formulations comprise a) 0.000001 to 10% by weight of at least one hydrophobin polypeptide sequence (i), b) 25 to 94.95% by weight of water, c) 5 to 50% by weight of surfactants, c) 0 to 5% by weight of a further conditioner, d) 0 to 10% by weight of further cosmetic constituents.

In the shampoo formulations it is possible to use all of the anionic, neutral, amphoteric or cationic surfactants customarily used in shampoos.

Suitable anionic surfactants are, for example, alkyl sulfates, alkyl ether sulfates, alkylsulfonates, alkylarylsulfonates, alkyl succinates, alkyl sulfosuccinates, N-alkoylsarcosinates, acyl taurates, acyl isothionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units, in the molecule.

Of suitability are, for example, sodium lauryl sulfate, ammonium lauryl sulfate, sodium lauryl ether sulfate, ammonium lauryl ether sulfate, sodium lauroyl sarcosinate, sodium oleyl succinate, ammonium lauryl sulfosuccinate, sodium dodecylbenzenesulfonate, triethanolamine dodecylbenzenesulfonate.

Suitable amphoteric surfactants are, for example, alkylbetaines, alkylamidopropyl-betaines, alkylsulfobetaines, alkyl glycinates, alkyl carboxyglycinates, alkyl amphoacetates or amphopropionates, alkylamphodiacetates or amphodipropionates.

For example, cocodimethylsulfopropylbetaine, laurylbetaine, cocamidopropylbetaine or sodium cocamphopropionate can be used.

Suitable nonionic surfactants are, for example, the reaction products of aliphatic alcohols or alkylphenols having 6 to 20 carbon atoms in the alkyl chain, which may be linear or branched, with ethylene oxide and/or propylene oxide. The amount of alkylene oxide is about 6 to 60 mol per mole of alcohol. In addition, alkylamine oxides, mono- or dialkylalkanolamides, fatty acid esters of polyethylene glycols, alkyl polyglycosides or sorbitan ether esters are suitable.

Furthermore, the shampoo formulations can comprise customary cationic surfactants, such as, for example, quaternary ammonium compounds, for example cetyltrimethylammonium chloride.

In the shampoo formulations, customary conditioners can be used in combination with the hydrophobin polypeptide sequences (i) to achieve certain effects.

These include, for example, the abovementioned cationic polymers with the INCl name Polyquaternium, in particular copolymers of vinylpyrrolidone/N-vinylimidazolium salts (Luviquat FC, Luviquat&commat, HM, Luviquat MS, Luviquat Care), copolymers of N-vinylpyrrolidone/dimethylaminoethyl methacrylate, quaternized with diethyl sulfate (Luviquat D PQ 11), copolymers of N-vinylcaprolactam/N-vinylpyrrolidone/N-vinylimidazolium salts (Luviquat D Hold), cationic cellulose derivatives (Polyquaternium-4 and -10), acrylamide copolymers (Polyquaternium-7). In addition, protein hydrolysates can be used, as well as conditioning substances based on silicone compounds, for example polyalkylsiloxanes, polyarylsiloxanes, polyarylalkylsiloxanes, polyethersiloxanes or silicone resins. Further suitable silicone compounds are dimethicone copolyols (CTFA) and aminofunctional silicone compounds such as amodimethicones (CTFA). In addition, cationic guar derivatives, such as guar hydroxypropyltrimonium chloride (INCl) can be used.

According to a further embodiment, this hair cosmetic or skin cosmetic preparation serves for the care or the protection of the skin or hair and is in the form of an emulsion, a dispersion, a suspension, an aqueous surfactant preparation, a milk, a lotion, a cream, a balsam, an ointment, a gel, granules, a powder, a stick preparation, such as, for example, a lipstick, a foam, an aerosol or a spray. Such formulations are highly suited for topical preparations. Suitable emulsions are oil-in-water emulsions and water-in-oil emulsions or microemulsions.

Generally, the hair cosmetic or skin cosmetic preparation is used for application to the skin (topically) or hair. Topical preparations here are understood as meaning those preparations which are suitable for applying the active substances to the skin in fine distribution and preferably in a form which can be absorbed by the skin. Of suitability for this purpose are, for example, aqueous and aqueous-alcoholic solutions, sprays, foams, foam aerosols, ointments, aqueous gels, emulsions of the O/W or W/O type, microemulsions or cosmetic stick preparations.

According to a preferred embodiment of the cosmetic composition according to the invention, the composition comprises a carrier. Preferred carriers are water, a gas, a water-based liquid, an oil, a gel, an emulsion or microemulsion, a dispersion or a mixture thereof. The carriers specified exhibit good compatibility with the skin. Of particular advantage for topical preparations are aqueous gels, emulsions or microemulsions.

Emulsifiers which can be used are nonionogenic surfactants, zwitterionic surfactants, ampholytic surfactants or anionic emulsifiers. The emulsifiers may be present in the composition according to the invention in amounts of from 0.1 to 10% by weight, preferably 1 to 5% by weight, based on the composition.

As nonionogenic surfactant it is possible to use, for example, a surfactant from at least one of the following groups:

Addition products of from 2 to 30 mol of ethylene oxide and/or 0 to 5 mol of propylene oxide onto linear fatty alcohols having 8 to 22 carbon atoms, onto fatty acids having 12 to 22 carbon atoms and onto alkylphenols having 8 to 15 carbon atoms in the alkyl group;

C_{12/18}-fatty acid mono- and diesters of addition products of from 1 to 30 mol of ethylene oxide onto glycerol;

glycerolmono- and diesters and sorbitanmono- and diesters of saturated and unsaturated fatty acids having 6 to 22 carbon atoms and ethylene oxide addition products thereof;
alkyl mono- and oligoglycosides having 8 to 22 carbon atoms in the alkyl radical and ethoxylated analogs thereof;
addition products of from 15 to 60 mol of ethylene oxide onto castor oil and/or hydrogenated castor oil;
polyol and, in particular, polyglycerol esters, such as, for example, polyglycerol polyricinoleate, polyglycerol poly-12-hydroxystearate or polyglycerol dimerate. Mixtures of compounds from two or more of these classes of substances are likewise suitable;
addition products of from 2 to 15 mol of ethylene oxide onto castor oil and/or hydrogenated castor oil;
partial esters based on linear, branched, unsaturated or saturated C_{6/22}-fatty acids, ricinoleic acid and 12-hydroxystearic acid and glycerol, polyglycerol, pentaerythritol, dipentaerythritol, sugar alcohols (e.g. sorbitol), alkyl glucosides (e.g. methyl glucoside, butyl glucoside, lauryl glucoside) and polyglucosides (e.g. cellulose);
mono-, di- and trialkyl phosphates, and mono-, di- and/or tri-PEG alkyl phosphates and salts thereof;
wool wax alcohols;
polysiloxane, polyalkyl, polyether copolymers and corresponding derivatives;
mixed esters of pentaerythritol, fatty acids, citric acid and fatty alcohol as in DE-C 1165574 and/or mixed esters of fatty acids having 6 to 22 carbon atoms, methylglucose and polyols, preferably glycerol or polyglycerol, and polyalkylene glycols and betaines.

In addition, emulsifiers which may be used are zwitterionic surfactants. Zwitterionic surfactants is the term used to describe those surface-active compounds which carry at least one quaternary ammonium group and at least one carboxylate group or a sulfonate group in the molecule. Particularly suitable zwitterionic surfactants are the so-called betaines, such as the N-alkyl-N,N-dimethylammonium glycinates, for example coconut alkyldimethylammonium glycinate, N-acylaminopropyl-N,N-dimethylammonium glycinates, for example coconut acylaminopropyldimethylammonium glycinate, and 2-alkyl-3-carboxylmethyl-3-hydroxyethylimidazolines having in each case 8 to 18 carbon atoms in the alkyl or acyl group, and the coconut acylaminoethyl hydroxyethylcarboxymethyl glycinate. Of particular preference is the fatty acid amide derivative known under the CTFA name Cocamidopropyl Betaine.

Likewise suitable emulsifiers are ampholytic surfactants. Ampholytic surfactants are understood as meaning those surface-active compounds which, apart from a C_{8,18}-alkyl or -acyl group in the molecule, comprise at least one free amino group and at least one —COOH or —SO₃H group and are capable of forming internal salts. Examples of suitable ampholytic surfactants are N-alkylglycines, N-alkylpropionic acids, N-alkylaminobutyric acids, N-alkyliminodipropionic acids, N-hydroxyethyl-N-alkylamido-propylglycines, N-alkyltaurines, N-alkylsarcosines, 2-alkylaminopropionic acids and alkylaminoacetic acids having in each case about 8 to 18 carbon atoms in the alkyl group.

Particularly preferred ampholytic surfactants are N-coconut-alkylaminopropionate, coconut acylaminoethylaminopropionate and C_{12/18}-acylsarcosine. Besides the ampholytic emulsifiers, quaternary emulsifiers are also suitable, particular preference being given to those of the ester quat type, preferably methyl-quaternized difatty acid triethanolamine ester salts. Furthermore, anionic emulsifiers which may be used are alkyl ether sulfates, monoglyceride sulfates, fatty acid sulfates, sulfosuccinates and/or ether carboxylic acids.

Suitable oil bodies are Guerbet alcohols based on fatty alcohols having 6 to 18, preferably 8 to 10, carbon atoms, esters of linear C₆-C₂₂-fatty acids with linear C₆-C₂₂-fatty alcohols, esters of branched C₆-C₁₃-carboxylic acids with linear C₆-C₂₂-fatty alcohols, esters of linear C₆-C₂₂-fatty acids with branched alcohols, in particular 2-ethylhexanol, esters of linear and/or branched fatty acids with polyhydric alcohols (such as, for example, propylene glycol, dimerdiol or trimertriol) and/or Guerbet alcohols, triglycerides based on C₆-C₁₀-fatty acids, liquid mono-/di-, triglyceride mixtures based on C₆-C₁₈-fatty acids, esters of C₆-C₂₂-fatty alcohols and/or Guerbet alcohols with aromatic carboxylic acids, in particular benzoic acid, esters of C₂-C₁₂-dicarboxylic acids with linear or branched alcohols having 1 to 22 carbon atoms or polyols having 2 to 10 carbon atoms and 2 to 6 hydroxyl groups, vegetable oils, branched primary alcohols, substituted cyclohexanes, linear C₆-C₂₂-fatty alcoholcarbonates, Guerbet carbonates, esters of benzoic acid with linear and/or branched C₆-C₂₂-alcohols (e.g. Finsolv® TN), dialkyl ethers, ring-opening products of epoxidized fatty acid esters with polyols, silicone oils and/or aliphatic or naphthenic hydrocarbons. Further oil bodies which may be used are also silicone compounds, for example dimethylpolysiloxanes, methyl-phenylpolysiloxanes, cyclic silicones, and amino-, fatty-acid-, alcohol-, polyether-, epoxy-, fluorine-, alkyl- and/or glycoside-modified silicone compounds which may be present at room temperature either as liquids or in resin form. The oil bodies may be present in the compositions according to the invention in amounts of from 1 to 90% by weight, preferably 5 to 80% by weight, and in particular 10 to 50% by weight, based on the composition.

By coupling corresponding compounds onto a hydrophobin polypeptide (i) it is possible to significantly prolong the active time on the skin. The coupling takes place as described above, formulation and application takes place by methods known to the person skilled in the art. For deodorants in particular, suitable effector molecules (ii) are: perfume oils, cyclodextrins, ion exchangers, zinc ricinoleate, antimicrobial/bacteriostatic compounds (e.g. DCMX, Irgasan DP 300, TCC).

Of suitability for antiperspirants are: tannins, and zinc/aluminum salts.

A further field of use for the substances according to the invention is the therapeutic or prophylactic use for certain diseases of the skin and mucosa. In the oral cavity, pharyngeal cavity and nasal cavity in particular it is advantageous to bind active substances for therapy/prophylaxis via a hydrophobin polypeptide sequence (i) to an increased and longer-lasting extent. Fields of use for this are, in particular:
viral diseases (e.g. Herpes, Coxsackie, Varicella zoster, Cytomegalovirus etc.)
bacterial diseases (e.g. TB, syphilis etc.)
fungal diseases (e.g. Candida, Cryptococcus, Histoplasmosis, Aspergillus, Mucormycosis etc.)
tumor diseases (e.g. melanoma, adenoma etc.)
autoimmune diseases (e.g. PEMPHIGUS VULGARIS, BULLOUS PEMPHIGOID, SYSTEMIC LUPUS ERYTHEMATOSIS etc.)
sunburn
parasitic attack (e.g. ticks, mites, fleas etc.)
insect contact (e.g. blood-sucking insects such as Anopheles etc.)

The substances suitable for therapy or prophylaxis (e.g. corticoids, immunosuppressing compounds, antibiotics, antimycotics, antiviral compounds, insect repellent etc.) can be coupled to the keratin-binding polypeptides (i) via the above-described linkers (a linker to be optimized depending on the functionality to be coupled).

### EXPERIMENTAL PART

### Example 1

### Preparation for the Cloning of yaad-His₆/yaaE-His₆

Using the oligonucleotides Hal570 and Hal571 (Hal 572/Hal 573), a polymerase chain reaction was carried out. The template DNA used was genomic DNA from the bacterium Bacillus subtilis. The PCR fragment obtained comprised the coding sequence of the gene yaaD/yaaE from Bacillus subtilis, and at the ends in each case an NcoI or BglII restriction cleavage site. The PCR fragment was purified and cleaved with the restriction endonucleases NcoI and BglII. This DNA fragment was used as insert, and cloned in the vector pQE60 from Qiagen which had been linearized beforehand with the restriction endonucleases NcoI and BglII. The vectors pQE60YAAD#2/pQE60YaaE#5 produced in this way can be used for the expression of proteins consisting of YAAD::HiS₆ and YAAE::HIS₆.

HaI570:
gcgcgcccatggctcaaacaggtactga
HaI571:
gcagatctccagccgcgttcttgcatac
HaI572:
ggccatgggattaacaataggtgtactagg
HaI573:
gcagatcttacaagtgccttttgcttatattcc

### Example 2

### Cloning of yaad-Hydrophobin DewA-His₆

Using the oligonucleotides KaM 416 and KaM 417, a polymerase chain reaction was carried out. The template DNA used was genomic DNA of the mold Aspergillus nidulans. The PCR fragment obtained comprised the coding sequence of the hydrophobin gene dewA and an N-terminal factor Xa proteinase cleavage site. The PCR fragment was purified and cut with the restriction endonuclease BamHI. This DNA fragment was used as insert and cloned into the vector pQE60YAAD#2 which had been linearized beforehand with the restriction endonuclease BgIll.

The vector #508 formed in this way can be used for the expression of a fusion protein consisting of YAAD::Xa::dewA::HIS₆.

KaM416:
GCAGCCCATCAGGGATCCCTCAGCCTTGGTACCAGCGC
KaM417:
CCCGTAGCTAGTGGATCCATTGAAGGCCGCATGAAGTTCTCCGTCTCCGC

### Example 3

### Cloning of yaad-Hydrophobin RodA-His₆

The plasmid #513 was cloned analogously to plasmid #508 using the oligonucleotides KaM 434 and KaM 435.

KaM434:
GCTAAGCGGATCCATTGAAGGCCGCATGAAGTTCTCCATTGCTGC
KaM435:
CCAATGGGGATCCGAGGATGGAGCCAAGGG

### Example 4

### Cloning of yaad-Hydrophobin BASF1-His₆

Plasmid #507 was cloned analogously to plasmid #508 using the oligonucleotides KaM 417 and KaM418.

The template DNA used was an artificially synthesized DNA sequence—hydrophobin BASF1 (see annex).

KaM417:
CCCGTAGCTAGTGGATCCATTGAAGGCCGCATGAAGTTCTCCGTCTCCGC
KaM418:
CTGCCATTCAGGGGATCCCATATGGAGGAGGGAGACAG

### Example 5

### Cloning of yaad-Hydrophobin BASF2-His₆

The plasmid #506 was cloned analogously to plasmid #508 using the oligonucleotides KaM 417 and KaM 418.

The template DNA used was an artificially synthesized DNA sequence—hydrophobin BASF2 (see annex).

KaM417:
CCCGTAGCTAGTGGATCCATTGAAGGCCGCATGAAGTTCTCCGTCTCCGC
KaM418:
CTGCCATTCAGGGGATCCCATATGGAGGAGGGAGACAG

### Example 6

### Cloning of yaad-Hydrophobin SC3-His₆

The plasmid #526 was cloned analogously to plasmid #508 using the oligonucleotides KaM464 and KaM465.

The template DNA used was cDNA from Schyzophyllum commune (see annex).

KaM464:
CGTTAAGGATCCGAGGATGTTGATGGGGGTGC
KaM465:
GCTAACAGATCTATGTTCGCCCGTCTCCCCGTCGT

### Example 7

### Fermentation of the Recombinant E. coli Strain yaad-Hydrophobin DewA-His₆

Inoculation of 3 ml of LB liquid medium with a yaad-hydrophobin DewA-His6 expressing E. coli strain in 15 ml Greiner tubes. Incubation for 8 h at 37° C. on a shaker at 200 rpm. 2 1 l Erlenmeyer flasks with baffles and 250 ml of LB medium (+100 μg/ml of ampicillin) are each inoculated with in each case 1 ml of the preculture and incubated for 9 h at 37° C. on a shaker with 180 rpm.

Inoculate 13.5 l of LB medium (+100 μg/ml of ampicillin) in a 20 l fermenter with 0.5 l preculture (OD600 nm measured 1:10 against H₂O). At an OD60 nm of ˜3.5 addition of 140 ml of 100 mM IPTG. After 3 h fermenter, cool to 10° C. and centrifuge off fermentation broth. Use cell pellet for further purification.

### Example 8

### Purification of the Recombinant Hydrophobin Fusion Protein

(Purification of Hydrophobin Fusion Proteins which have a C-Terminal His6 Tag)

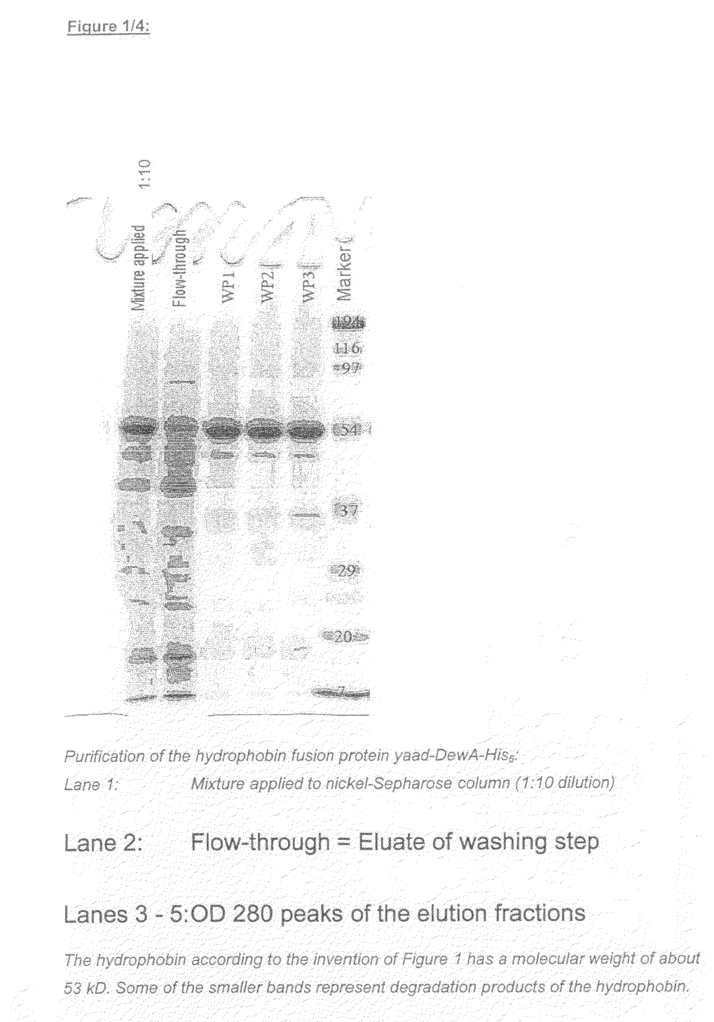

100 g of cell pellet (100-500 mg of hydrophobin) are made up to a total volume of 200 ml with 50 mM sodium phosphate buffer, pH 7.5 and resuspended. The suspension is treated with an Ultraturrax model T25 (Janke and Kunkel; IKA-Labortechnik) for 10 minutes and then incubated for 1 hour at room temperature with 500 units of benzonase (Merck, Darmstadt; Order No. 1.01697.0001) to degrade the nucleic acids. Prior to cell disruption, the mixture is filtered using a glass cartridge (P1). For cell disruption and for shearing the remaining genomic DNA, two homogenizer runs are carried out at 1500 bar (Microfluidizer M-110EH; Microfluidics Corp.). The homogenate is centrifuged (Sorvall RC-5B, GSA-Rotor, 250 ml centrifuge beaker, 60 minutes, 4° C., 12 000 rpm, 23 000 g), the supernatant is put on ice and the pellet is resuspended in 100 ml of sodium phosphate buffer, pH 7.5. Centrifugation and resuspension are repeated three times, the sodium phosphate buffer comprising 1% SDS at the third repeat. Following resuspension, the mixture is stirred for one hour and a final centrifugation is carried out (Sorvall RC-5B, GSA-Rotor, 250 ml centrifuge beaker, 60 minutes, 4° C., 12 000 rpm, 23 000 g). According to SDS-PAGE analysis, the hydrophobin is present in the supernatant after the final centrifugation (FIG. 1). The experiments show that the hydrophobin is present in the corresponding E. coli cells probably in the form of inclusion bodies. 50 ml of the hydrophobin-comprising supernatant are applied to a 50 ml nickel-Sepharose High Performance 17-5268-02 column (Amersham) which has been equilibrated using 50 mM Tris-Cl pH 8.0 buffer. The column is washed with 50 mM Tris-Cl pH 8.0 buffer and the hydrophobin is then eluted using 50 mM Tris-Cl pH 8.0 buffer which comprises 200 mM imidazole. To remove the imidazole, the solution is dialyzed against 50 mM Tris-Cl pH 8.0 buffer.

FIG. 1 shows the purification of the inventive hydrophobin:

| | |
|---|---|
| Lane 1: | Mixture applied to nickel-Sepharose column (1:10 dilution) |
| Lane 2: | Flow-through = eluate of washing step |
| Lanes 3-5: | OD 280 peaks of the elution fractions |

The hydrophobin according to the invention in FIG. 1 has a molecular weight of about 53 kD. Some of the smaller bands represent degradation products of the hydrophobin.

### Example 9

### Coating/Evaluation of Surfaces with Hydrophobin

The evaluation of the coating properties of hydrophobin and hydrophobin fusion protein is preferably carried out on glass or Teflon as models for a hydrophilic or hydrophobic surface.

### Standard Experiments for Coating

### Glass:

concentration of hydrophobin: 1-100 μg/ml
incubation of glass slides overnight (temperature: 80° C.) in 50 mM Na acetate pH4+0.1% Tween 20
after coating, wash in demineralized water
then incubation 10 min/80° C./1% SDS
wash in demineralized water

### Teflon:

concentration: 1-100 μg/ml
Incubation of Teflon plates overnight (temperature: 80° C.) in 10 mM tris pH 8
after coating, wash in demineralized water
incubation 10 min/80° C./0.1% Tween 20
wash in demineralized water
then incubation 10 min/80° C./1% SDS
wash in demineralized water

The samples are dried in air and the contact angle (in degrees) of a drop of 5 μl of water is determined. The following values are, for example, obtained:

Mixture with yaad-DewA fusion protein as in Example 8 (control: without protein; yaad-dewA-his₆:100 μg/ml of purified fusion partner):

after 1%
SDS 80° C.

| | Teflon | Glass |
|---|---|---|
| Control | 96.8 | 30 |
| yaad | 97.4 | 38.7 |
| 100 μg/ml | 77.7 | 76.8 |
| 50 μg/ml | 85.9 | 77.9 |
| 10 μg/ml | 83.5 | 74.5 |
| 5 μg/ml | 104 | 70.3 |
| 1 μg/ml | 104.9 | 73 |

### Example 10

### Coating/Evaluation of Surfaces with Hydrophobin

Glass (window glass, Süddeutsche Glas, Mannheim):
concentration of hydrophobin: 100 μg/ml
incubation of glass plates overnight (temperature 80° C.) in 50 mM Na acetate pH 4+0.1% Tween 20
after coating, wash in distilled water
then incubation 10 min/80° C./1% SDS solution in dist. water
wash in dist. water

The samples are dried in air and the contact angle (in degrees) of a drop of 5 μl of water is determined.

The contact angle measurement was determined using a Dataphysics Contact Angle System OCA 15+, software SCA 20.2.0. (November 2002). The measurement was carried out in accordance with the manufacturer's instructions.

Untreated glass gave a contact angle of 30±5°; coating with a functional hydrophobin as in Example 8 (yaad-dewA-his₆) gave contact angles of 75±5°.

### Example 11

### Binding to Skin 1

### Qualitative

A visual qualitative test was developed in order to test whether hydrophobin binds to skin.

### Solutions Used:

Blocking solution: DIG Wash+Bufferset 1585762 Boehringer MA (10× solution) diluted in TBS

### TBS: 20 mM Tris; 150 mM NaCl pH 7.5

### TTBS: TBS+0.05% Tween 20

The first step is the transfer of the external keratin layer from the skin onto a stable carrier. For this, a transparent adhesive strip was applied firmly to depilated human skin and removed again. The test can be carried out directly on the transparent adhesive strip, or the adhering keratin layer can be transferred to a glass slide by sticking once again. The demonstration of binding was undertaken as follows:
for incubation with the various reagents, transfer to a Falcon vessel
if appropriate addition of ethanol for degreasing, removal of ethanol and drying of the slide
incubation for 1 h at room temperature with blocking buffer
washing for 2×5 min with TTBS
washing for 1×5 min with TBS
incubation with the hydrophobin to be tested (coupled to tag—e.g. His₆, HA etc.) or control protein in TBS/0.05% Tween 20 for 24 h at room temperature
removal of the supernatant
3× washing with TBS
incubation for 1 h at room temperature with monoclonal anti-poly-histidine antibodies, diluted 1:2000 in TBS+0.01% blocking
washing for 2×5 min with TTBS
washing for 1×5 min with TBS
incubation for 1 h at room temperature with anti-mouse IgG alkaline phosphatase conjugate, diluted 1:5000 in TBS+0.01% blocking
washing for 2×5 min with TTBS
washing for 1×5 min with TBS
addition of phosphatase substrate (NBT-BCIP; Boehringer MA 1 tablet/40 ml of water 2.5 min; stop:with water)
optical detection of the colored precipitate with the naked eye or using a microscope. A blue colored precipitate indicates that hydrophobin has bound to the skin.

### Example 12

### Binding to Skin 2

### Quantitative

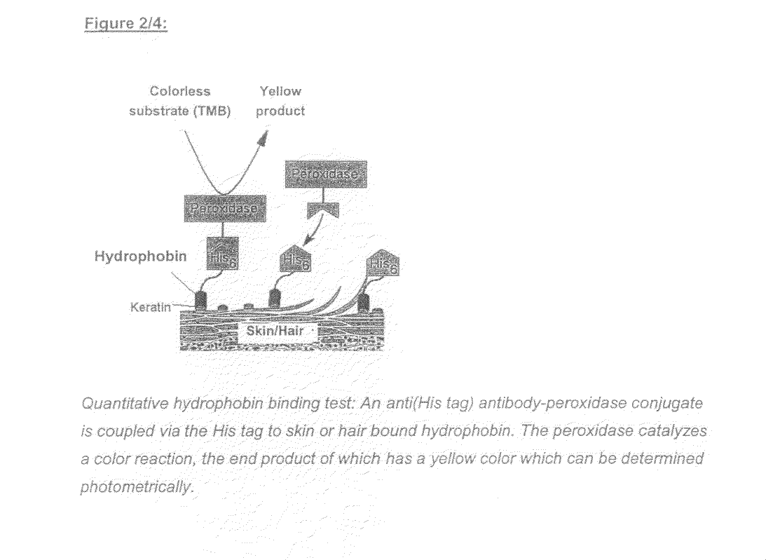

A quantitative test was developed which allows the hair/skin binding strength of the hydrophobin to be compared with non-specific proteins (FIG. 2).

A 5 mm cork borer was used to bore out a section from a thawed dry piece of skin without hair (human or pig) (or in case of a surface test a section of skin was inserted into a Falcon lid). The skin sample was then converted to a thickness of 2-3 mm in order to remove any tissue present. The skin sample was then transferred to an Eppendorf vessel (protein low-bind) in order to carry out the binding demonstration (see also FIG. 2):
2× washing with PBS/0.05% Tween 20
addition of 1 ml of 1% BSA in PBS and incubation for 1 h at room temperature, gentle swinging movements (900 rpm).
removal of the supernatant
addition of 100 μg of hydrophobin in PBS+0.05% Tween 20; incubation for 2 h at room temperature and gentle swinging movements (900 rpm).
removal of the supernatant
3× washing with PBS/0.05% Tween 20
incubation with 1 ml of monoclonal mouse anti-tag (His6 or HA or specific KBD) antibodies with peroxidase conjugate (1:2000 in PBS/0.05% Tween 20) [Monoclonal AntipolyHistidin Peroxidase Conjugate, produced in mouse, lyophilized powder, Sigma] for 24 h at room temperature, gentle swinging movement (900 rpm)
3× washing with PBS/0.05% Tween 20
addition of peroxidase substrate (1 ml/Eppendorf vessel; composition see below)
allow reaction to proceed until a blue coloration is obtained (about 1:30 minutes).
stop the reaction with 100 μl of 2 M H₂SO₄.
the absorption was measured at 405 nm

Peroxidase Substrate (Prepared Shortly Beforehand):

0.1 ml of TMB solution (42 mM TMB in DMSO)

+10 ml substrate buffer (0.1 M sodium acetate pH 4.9)

+14.7 μl H₂O₂ 3% strength

### Example 13

### Binding to Hair

### Quantitative

In order to be able to also demonstrate the binding strength of the hydrophobin to hair compared to other proteins, a quantitative assay was developed (FIG. 2). In this test, hair was firstly incubated with hydrophobin and excess hydrophobin was washed off. An antibody-peroxidase conjugate was then coupled via the His tag of the hydrophobin. Nonbound antibody-peroxidase conjugate was washed off again. The bound antibody-peroxidase conjugate [Monoclonal Antipoly-Histidin Peroxidase Conjugate, produced in mouse, lyophilized powder, Sigma] can convert a colorless substrate (TMB) into a colored product, which was measured photometrically at 405 nm. The intensity of the absorption indicates the amount of bound hydrophobin or comparison protein. The comparison protein chosen was, for example, YaaD from B. subtilis which likewise had—as is necessary for this test—a His tag for the detection. Instead of the His tag it is also possible to use other specific antibodies conjugated with peroxidase.

5 mg of hair (human) are cut into sections 5 mm in length and transferred to Eppendorf vessels (protein low-bind) in order to carry out the binding demonstration:
addition of 1 ml of ethanol for degreasing
centrifugation, removal of ethanol and washing of the hair with H₂O
addition of 1 ml of 1% BSA in PBS and incubation for 1 h at room temperature, gentle swinging movements.
centrifugation, removal of the supernatant
addition of the hydrophobin to be tested (coupled on tag—e.g. His₆, HA etc.) or control protein in 1 ml of PBS/0.05% Tween 20; incubation for 16 h at 4° C. (or at least 2 h at room temperature) with gentle swinging movements.
centrifugation, removal of the supernatant
3× washing with PBS/0.05% Tween 20
incubation with 1 ml of monoclonal mouse anti-tag (His6 or HA) antibodies with peroxidase conjugate (1:2000 in PBS/0.05% Tween 20) [Monoclonal AntipolyHistidin Peroxidase Conjugate, produced in mouse, lyophilized powder, Sigma] for 24 h at room temperature, gentle swinging movement
3× washing with PBS/0.05% Tween 20
addition of peroxidase substrate (1 ml/Eppendorf vessel)
allow the reaction to proceed until a blue coloration is obtained (about 2 minutes).
Stop the reaction with 100 μl of 2 M H₂SO₄.
The absorption is measured at 405 nm

Peroxidase Substrate (Prepared Shortly Beforehand):

0.1 ml TMB solution (42 mM TMB in DMSO)

+10 ml substrate buffer (0.1 M sodium acetate pH 4.9)

+14.7 μl H₂O₂ 3% strength

BSA=Bovine serum albumin

PBS=Phosphate buffered salt solution

Tween 20=Polyoxyethylene sorbitan monolaureate, n about 20

TMB=3,5,3′,5′-tetramethylbenzidine

A binding test on hair carried out by way of example for hydrophobin demonstrated considerable superiority of the binding of hydrophobin to hair compared with significantly poorer binding of the comparison protein YaaD:

TABLE 1
Quantitative hydrophobin activity test on hair: 1) Buffer;
2) Comparison protein yaad; 3) Hydrophobin. The table
shows the measured absorption values at 405 nm.

| | | |
|---|---|---|
| 1 | Buffer | A_{405 nm} = 0.05 |
| 2 | Comparison protein yaad | A_{405 nm} = 0.12 |
| 3 | Hydrophobin | A_{405 nm} = 1.43 |

Analogously to Example 11 (binding to skin), the binding to mucosa can also be measured by removing a sample of mucosa (for example human oral mucosa) using a transparent adhesive strip, which can then be investigated with regard to binding effect.

The binding to teeth can be determined by incubating the polypeptide sequences to be investigated directly with the tooth surface (for example cow teeth) and carrying out measurements according to Example 11.

### Example 14

### Derivatization of Hydrophobin with “Alexa” Dye and Binding to Hair

One way of coupling active substances or effect substances to proteins (general principle FIG. 3) is via the SH groups of the cysteines. Prior to coupling the dye Alexa Fluor 532, the disulfide bridges of the hydrophobin are cleaved:

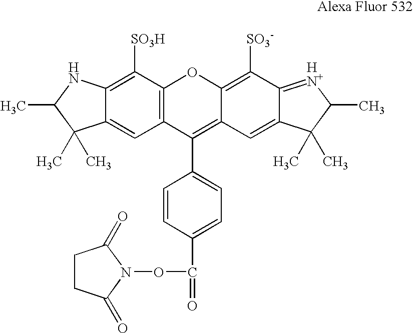

1 mg hydrophobin

0.5 ml buffer (75 mM Tris pH 8.0
2.5 mM EDTA
1 mM DTT)

incubation for 30 minutes at 37° C.

The dye is coupled in accordance with manufacturer's instructions (Alexa 532 Protein Labeling Kit; Molecular Probes; MP-A-10236)

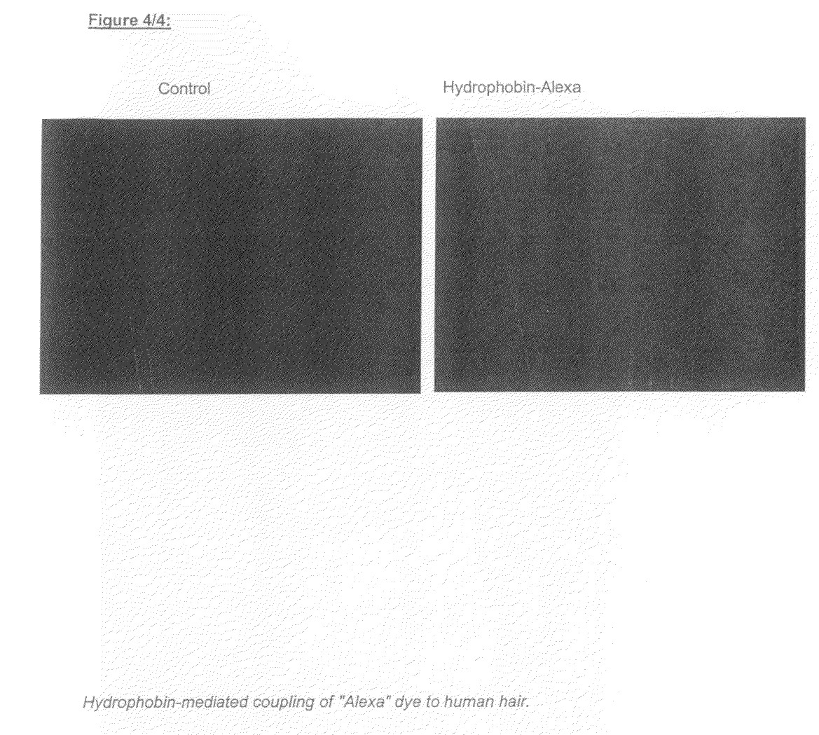

The coating of human hair with Alexa-coupled hydrophobin is carried out as follows:
incubate 10 mg of human hair with 50 μg/ml of Alexa hydrophobin or control protein yaad or uncoupled dye Alexa 532 in buffer TBS for 24 hours at room temperature
2× washing with TBS/0.05% Tween 20
1× washing with TBS
1× washing with TBS/1% SDS
detection in a fluorescence microscope (FIG. 4)

### Example 15

### Use of the Hydrophobin in an Emulsion for Daycare

### O/W Type

### WS 1%:

| | % | Ingredients (INCI) |
|---|---|---|
| A | 1.7 | Ceteareth-6, Stearyl Alcohol |
| | 0.7 | Ceteareth-25 |
| | 2.0 | Diethylamino Hydroxybenzoyl Hexyl Benzoate |
| | 2.0 | PEG-14 Dimethicone |
| | 3.6 | Cetearyl Alcohol |
| | 6.0 | Ethylhexyl Methoxycinnamate |
| | 2.0 | Dibutyl Adipate |
| B | 5.0 | Glycerin |
| | 0.2 | Disodium EDTA |
| | 1.0 | Panthenol |
| | q.s. | Preservative |
| | 67.8 | Aqua dem. |
| C | 4.0 | Caprylic/Capric Triglyceride, Sodium Acrylates Copolymer |
| D | 0.2 | Sodium Ascorbyl Phosphate |
| | 1.0 | Tocopheryl Acetate |
| | 0.2 | Bisabolol |
| | 1.0 | Caprylic/Capric Triglyceride, Sodium Ascorbate, Tocopherol, |
| | | Retinol |
| | 1.0 | Aqueous solution with about 5% hydrophobin |
| E | q.s. | Sodium Hydroxide |

### WS 5%:

| | % | Ingredients (INCI) |
|---|---|---|
| A | 1.7 | Ceteareth-6, Stearyl Alcohol |
| | 0.7 | Ceteareth-25 |
| | 2.0 | Diethylamino Hydroxybenzoyl Hexyl Benzoate |
| | 2.0 | PEG-14 Dimethicone |
| | 3.6 | Cetearyl Alcohol |
| | 6.0 | Ethylhexyl Methoxycinnamate |
| | 2.0 | Dibutyl Adipate |
| B | 5.0 | Glycerin |
| | 0.2 | Disodium EDTA |
| | 1.0 | Panthenol |
| | q.s. | Preservative |
| | 63.8 | Aqua dem. |
| C | 4.0 | Caprylic/Capric Triglyceride, Sodium Acrylates Copolymer |
| D | 0.2 | Sodium Ascorbyl Phosphate |
| | 1.0 | Tocopheryl Acetate |
| | 0.2 | Bisabolol |
| | 1.0 | Caprylic/Capric Triglyceride, Sodium Ascorbate, Tocopherol, |
| | | Retinol |
| | 5.0 | Aqueous solution with about 5% hydrophobin |
| E | q.s. | Sodium Hydroxide |

Preparation: Heat phases A and B separately from one another to about 80° C. Stir phase B into phase A and homogenize. Stir phase C into the combined phases A and B and homogenize again. Cool to about 40° C. with stirring, add phase D, adjust the pH to about 6.5 with phase E, homogenize and cool to room temperature with stirring.

Note: The formulation is prepared without protective gas. Bottling must take place in oxygen-impermeable packagings, e.g. aluminum tubes.

### Example 16

### Use of the Hydrophobin in a Protective Day Cream

### O/W Type

### WS 1%:

| | % | Ingredients (INCI) |
|---|---|---|
| A | 1.7 | Ceteareth-6, Stearyl Alcohol |
| | 0.7 | Ceteareth-25 |
| | 2.0 | Diethylamino Hydroxybenzoyl Hexyl Benzoate |
| | 2.0 | PEG-14 Dimethicone |
| | 3.6 | Cetearyl Alcohol |
| | 6.0 | Ethylhexyl Methoxycinnamate |
| | 2.0 | Dibutyl Adipate |
| B | 5.0 | Glycerin |
| | 0.2 | Disodium EDTA |
| | 1.0 | Panthenol |
| | q.s. | Preservative |
| | 68.6 | Aqua dem. |
| C | 4.0 | Caprylic/Capric Triglyceride, Sodium Acrylates Copolymer |
| D | 1.0 | Sodium Ascorbyl Phosphate |
| | 1.0 | Tocopheryl Acetate |
| | 0.2 | Bisabolol |
| | 1.0 | Aqueous solution with about 5% hydrophobin |
| E | q.s. | Sodium Hydroxide |

### WS 5%:

| | % | Ingredients (INCI) |
|---|---|---|
| A | 1.7 | Ceteareth-6, Stearyl Alcohol |
| | 0.7 | Ceteareth-25 |
| | 2.0 | Diethylamino Hydroxybenzoyl Hexyl Benzoate |
| | 2.0 | PEG-14 Dimethicone |
| | 3.6 | Cetearyl Alcohol |
| | 6.0 | Ethylhexyl Methoxycinnamate |
| | 2.0 | Dibutyl Adipate |
| B | 5.0 | Glycerin |
| | 0.2 | Disodium EDTA |
| | 1.0 | Panthenol |
| | q.s. | Preservative |
| | 64.6 | Aqua dem. |
| C | 4.0 | Caprylic/Capric Triglyceride, Sodium Acrylates Copolymer |
| D | 1.0 | Sodium Ascorbyl Phosphate |
| | 1.0 | Tocopheryl Acetate |
| | 0.2 | Bisabolol |
| | 5.0 | Aqueous solution with about 5% hydrophobin |
| E | q.s. | Sodium Hydroxide |

Preparation: Heat phases A and B separately from one another to about 80° C. Stir phase B into phase A and homogenize. Incorporate phase C into the combined phases A and B and homogenize. Cool to about 40° C. with stirring. Add phase D, adjust the pH to about 6.5 with phase E and homogenize. Cool to room temperature with stirring.

### Example 17

### Use of the Hydrophobin in a Face-Cleansing Lotion

### O/W Type

### WS 1%:

| | % | Ingredients (INCI) |
|---|---|---|
| A | 10.0 | Cetearyl Ethylhexanoate |
| | 10.0 | Caprylic/Capric Triglyceride |
| | 1.5 | Cyclopentasiloxane, Cyclohexasilosane |
| | 2.0 | PEG-40 Hydrogenated Castor Oil |
| B | 3.5 | Caprylic/Capric Triglyceride, Sodium Acrylates Copolymer |
| C | 1.0 | Tocopheryl Acetate |
| | 0.2 | Bisabolol |
| | q.s. | Preservative |
| | q.s. | Perfume oil |
| D | 3.0 | Polyquaternium-44 |
| | 0.5 | Cocotrimonium Methosulfate |
| | 0.5 | Ceteareth-25 |
| | 2.0 | Panthenol, Propylene Glycol |
| | 4.0 | Propylene Glycol |
| | 0.1 | Disodium EDTA |
| | 1.0 | Aqueous solution with about 5% hydrophobin |
| | 60.7 | Aqua dem. |

### WS 5%:

| | % | Ingredients (INCI) |
|---|---|---|
| A | 10.0 | Cetearyl Ethylhexanoate |
| | 10.0 | Caprylic/Capric Triglyceride |
| | 1.5 | Cyclopentasiloxane, Cyclohexasilosane |
| | 2.0 | PEG-40 Hydrogenated Castor Oil |
| B | 3.5 | Caprylic/Capric Triglyceride, Sodium Acrylates Copolymer |
| C | 1.0 | Tocopheryl Acetate |
| | 0.2 | Bisabolol |
| | q.s. | Preservative |
| | q.s. | Perfume oil |
| D | 3.0 | Polyquaternium-44 |
| | 0.5 | Cocotrimonium Methosulfate |
| | 0.5 | Ceteareth-25 |
| | 2.0 | Panthenol, Propylene Glycol |
| | 4.0 | Propylene Glycol |
| | 0.1 | Disodium EDTA |
| | 5.0 | Aqueous solution with about 5% hydrophobin |
| | 56.7 | Aqua dem. |

Preparation: Dissolve phase A. Stir phase B into phase A, incorporate phase C into the combined phases A and B. Dissolve phase D, stir into the combined phases A, B and C and homogenize. After-stir for 15 min.

### Example 18

### Use of the hydrophobin in a Daily Care Body Spray

### WS 1%:

| | % | Ingredients (INCI) |
|---|---|---|
| A | 3.0 | Ethylhexyl Methoxycinnamate |
| | 2.0 | Diethylamino Hydroxybenzoyl Hexyl Benzoate |
| | 1.0 | Polyquaternium-44 |
| | 3.0 | Propylene Glycol |
| | 2.0 | Panthenol, Propylene Glycol |
| | 1.0 | Cyclopentasiloxane, Cyclohexasiloxane |
| | 10.0 | Octyldodecanol |
| | 0.5 | PVP |
| | 10.0 | Caprylic/Capric Triglyceride |
| | 3.0 | C12-15 Alkyl Benzoate |
| | 3.0 | Glycerin |
| | 1.0 | Tocopheryl Acetate |
| | 0.3 | Bisabolol |
| | 1.0 | Aqueous solution with about 5% hydrophobin |
| | 59.2 | Alcohol |

### WS 5%:

| | % | Ingredients (INCI) |
|---|---|---|
| A | 3.0 | Ethylhexyl Methoxycinnamate |
| | 2.0 | Diethylamino Hydroxybenzoyl Hexyl Benzoate |
| | 1.0 | Polyquaternium-44 |
| | 3.0 | Propylene Glycol |
| | 2.0 | Panthenol, Propylene Glycol |
| | 1.0 | Cyclopentasiloxane, Cyclohexasiloxane |
| | 10.0 | Octyldodecanol |
| | 0.5 | PVP |
| | 10.0 | Caprylic/Capric Triglyceride |
| | 3.0 | C12-15 Alkyl Benzoate |
| | 3.0 | Glycerin |
| | 1.0 | Tocopheryl Acetate |
| | 0.3 | Bisabolol |
| | 5.0 | Aqueous solution with about 5% hydrophobin |
| | 55.2 | Alcohol |

Preparation: Weigh in the components of phase A and dissolve to give a clear solution.

### Example 19

### Use of the Hydrophobin in a Skincare Gel

### WS 1%:

| | % | Ingredients (INCI) |
|---|---|---|
| A | 3.6 | PEG-40 Hydrogenated Castor Oil |
| | 15.0 | Alcohol |
| | 0.1 | Bisabolol |
| | 0.5 | Tocopheryl Acetate |
| | q.s. | Perfume oil |
| B | 3.0 | Panthenol |
| | 0.6 | Carbomer |
| | 1.0 | Aqueous solution with about 5% hydrophobin |
| | 75.4 | Aqua dem. |
| C | 0.8 | Triethanolamine |

### WS 5%:

| | % | Ingredients (INCI) |
|---|---|---|
| A | 3.6 | PEG-40 Hydrogenated Castor Oil |
| | 15.0 | Alcohol |
| | 0.1 | Bisabolol |
| | 0.5 | Tocopheryl Acetate |
| | q.s. | Perfume oil |
| B | 3.0 | Panthenol |
| | 0.6 | Carbomer |
| | 5.0 | Aqueous solution with about 5% hydrophobin |
| | 71.4 | Aqua dem. |
| C | 0.8 | Triethanolamine |

Preparation: Dissolve phase A to give a clear solution. Allow phase B to swell and neutralize with phase C. Stir phase A into the homogenized phase B and homogenize.

### Example 20

### Use of the Hydrophobin in an after Shave Lotion

### WS 1%:

| | % | Ingredients (INCI) |
|---|---|---|
| A | 10.0 | Cetearyl Ethylhexanoate |
| | 5.0 | Tocopheryl Acetate |
| | 1.0 | Bisabolol |
| | 0.1 | Perfume oil |
| | 0.3 | Acrylates/C10-30 Alkyl Acrylate Crosspolymer |
| B | 15.0 | Alcohol |
| | 1.0 | Panthenol |
| | 3.0 | Glycerin |
| | 1.0 | Aqueous solution with about 5% hydrophobin |
| | 0.1 | Triethanolamine |
| | 63.5 | Aqua dem. |

### WS 5%:

| | % | Ingredients (INCI) |
|---|---|---|
| A | 10.0 | Cetearyl Ethylhexanoate |
| | 5.0 | Tocopheryl Acetate |
| | 1.0 | Bisabolol |
| | 0.1 | Perfume oil |
| | 0.3 | Acrylates/C10-30 Alkyl Acrylate Crosspolymer |
| B | 15.0 | Alcohol |
| | 1.0 | Panthenol |
| | 3.0 | Glycerin |
| | 5.0 | Aqueous solution with about 5% hydrophobin |
| | 0.1 | Triethanolamine |
| | 59.5 | Aqua dem. |

Preparation: Mix the components of phase A. Dissolve phase B, incorporate into phase A and homogenize.

### Example 21

### Use of the Hydrophobin in an after Sun Lotion

### WS 1%:

| | % | Ingredients (INCI) |
|---|---|---|
| A | 0.4 | Acrylates/C10-30 Alkyl Acrylate Crosspolymer |
| | 15.0 | Cetearyl Ethylhexanoate |
| | 0.2 | Bisabolol |
| | 1.0 | Tocopheryl Acetate |
| | q.s. | Perfume oil |
| B | 1.0 | Panthenol |
| | 15.0 | Alcohol |
| | 3.0 | Glycerin |
| | 1.0 | Aqueous solution with about 5% hydrophobin |
| | 63.2 | Aqua dem. |
| C | 0.2 | Triethanolamine |

### WS 5%:

| | % | Ingredients (INCI) |
|---|---|---|
| A | 0.4 | Acrylates/C10-30 Alkyl Acrylate Crosspolymer |
| | 15.0 | Cetearyl Ethylhexanoate |
| | 0.2 | Bisabolol |
| | 1.0 | Tocopheryl Acetate |
| | q.s. | Perfume oil |
| B | 1.0 | Panthenol |
| | 15.0 | Alcohol |
| | 3.0 | Glycerin |
| | 5.0 | Aqueous solution with about 5% hydrophobin |
| | 59.2 | Aqua dem. |
| C | 0.2 | Triethanolamine |

Preparation: Mix the components of phase A. Stir phase B into phase A with homogenization. Neutralize with phase C and homogenize again.

### Example 22

### Use of the Hydrophobin in a Sunscreen Lotion

### WS 1%:

| | % | Ingredients (INCI) |
|---|---|---|
| A | 4.5 | Ethylhexyl Methoxycinnamate |
| | 2.0 | Diethylamino Hydroxybenzoyl Hexyl Benzoate |
| | 3.0 | Octocrylene |
| | 2.5 | Di-C12-13 Alkyl Malate |
| | 0.5 | Tocopheryl Acetate |
| | 4.0 | Polyglyceryl-3 Methyl Glucose Distearate |
| B | 3.5 | Cetearyl Isononanoate |
| | 1.0 | VP/Eicosene Copolymer |
| | 5.0 | Isohexadecane |
| | 2.5 | Di-C12-13 Alkyl Malate |
| | 3.0 | Titanium Dioxide, Trimethoxycaprylylsilane |
| C | 5.0 | Glycerin |
| | 1.0 | Sodium Cetearyl Sulfate |
| | 0.5 | Xanthan Gum |
| | 59.7 | Aqua dem. |
| D | 1.0 | Aqueous solution with about 5% hydrophobin |
| | 1.0 | Phenoxyethanol, Methylparaben, Ethylparaben, |
| | | Butylparaben, Propylparaben, |
| | | Isobutylparaben |
| | 0.3 | Bisabolol |

### WS 5%:

| | % | Ingredients (INCI) |
|---|---|---|
| A | 4.5 | Ethylhexyl Methoxycinnamate |
| | 2.0 | Diethylamino Hydroxybenzoyl Hexyl Benzoate |
| | 3.0 | Octocrylene |
| | 2.5 | Di-C12-13 Alkyl Malate |
| | 0.5 | Tocopheryl Acetate |
| | 4.0 | Polyglyceryl-3 Methyl Glucose Distearate |
| B | 3.5 | Cetearyl Isononanoate |
| | 1.0 | VP/Eicosene Copolymer |
| | 5.0 | Isohexadecane |
| | 2.5 | Di-C12-13 Alkyl Malate |
| | 3.0 | Titanium Dioxide, Trimethoxycaprylylsilane |
| C | 5.0 | Glycerin |
| | 1.0 | Sodium Cetearyl Sulfate |
| | 0.5 | Xanthan Gum |
| | 55.7 | Aqua dem. |
| D | 5.0 | Aqueous solution with about 5% hydrophobin |
| | 1.0 | Phenoxyethanol, Methylparaben, Ethylparaben, |
| | | Butylparaben, Propylparaben, |
| | | Isobutylparaben |
| | 0.3 | Bisabolol |

Preparation: Heat the components of phases A and B separately from one another to about 80° C. Stir phase B into phase A and homogenize. Heat phase C to about 80° C. and stir into the combined phases A and B with homogenization. Cool to about 40° C. with stirring, add phase D and homogenize again.

### Example 23

### Use of the Hydrophobin in a Sunscreen Lotion

### O/W Type

### WS 1%:

| | % | Ingredients (INCI) |
|---|---|---|
| A | 2.0 | Ceteareth-6, Stearyl Alcohol |
| | 2.0 | Ceteareth-25 |
| | 3.0 | Tribehenin |
| | 2.0 | Cetearyl Alcohol |
| | 2.0 | Cetearyl Ethylhexanoate |
| | 5.0 | Ethylhexyl Methoxycinnamate |
| | 1.0 | Ethylhexyl Triazone |
| | 1.0 | VP/Eicosene Copolymer |
| | 7.0 | Isopropyl Myristate |
| B | 5.0 | Zinc Oxide, Triethoxycaprylylsilane |
| C | 0.2 | Xanthan Gum |
| | 0.5 | Hydroxyethyl Acrylate/Sodium Acryloyldimethyl Taurate |
| | | Copolymer, Squalane, Polysorbate 60 |
| | 0.2 | Disodium EDTA |
| | 5.0 | Propylene Glycol |
| | 0.5 | Panthenol |
| | 60.9 | Aqua dem. |
| D | 1.0 | Aqueous solution with about 5% hydrophobin |
| | 0.5 | Phenoxyethanol, Methylparaben, Butylparaben, |
| | | Ethylparaben, Propylparaben, Isopropylparaben |
| | 1.0 | Tocopheryl Acetate |
| | 0.2 | Bisabolol |

### WS 5%:

| | % | Ingredients (INCI) |
|---|---|---|
| A | 2.0 | Ceteareth-6, Stearyl Alcohol |
| | 2.0 | Ceteareth-25 |
| | 3.0 | Tribehenin |
| | 2.0 | Cetearyl Alcohol |
| | 2.0 | Cetearyl Ethylhexanoate |
| | 5.0 | Ethylhexyl Methoxycinnamate |
| | 1.0 | Ethylhexyl Triazone |
| | 1.0 | VP/Eicosene Copolymer |
| | 7.0 | Isopropyl Myristate |
| B | 5.0 | Zinc Oxide, Triethoxycaprylylsilane |
| C | 0.2 | Xanthan Gum |
| | 0.5 | Hydroxyethyl Acrylate/Sodium Acryloyldimethyl Taurate |
| | | Copolymer, Squalane, Polysorbate 60 |
| | 0.2 | Disodium EDTA |
| | 5.0 | Propylene Glycol |
| | 0.5 | Panthenol |
| | 56.9 | Aqua dem. |
| D | 5.0 | Aqueous solution with about 5% hydrophobin |
| | 0.5 | Phenoxyethanol, Methylparaben, Butylparaben, |
| | | Ethylparaben, Propylparaben, Isopropylparaben |
| | 1.0 | Tocopheryl Acetate |
| | 0.2 | Bisabolol |

Preparation: Heat phase A to about 80° C. Stir in phase B and homogenize for 3 min. Likewise heat phase C to 80° C. and stir into the combined phases A and B with homogenization. Cool to about 40° C., stir in phase D and homogenize again.

### Example 24

### Use of the Hydrophobin in a Sunscreen Lotion

### O/W Type

### WS 1%:

| | % | Ingredients (INCI) |
|---|---|---|
| A | 3.5 | Ceteareth-6, Stearyl Alcohol |
| | 1.5 | Ceteareth-25 |
| | 7.5 | Ethylhexyl Methoxycinnamate |
| | 2.0 | Diethylamino Hydroxybenzoyl Hexyl Benzoate |
| | 2.0 | Cyclopentasiloxane, Cyclohexasiloxane |
| | 0.5 | Bees Wax |
| | 3.0 | Cetearyl Alcohol |
| | 10.0 | Caprylic/Capric Triglyceride |
| B | 5.0 | Titanium Dioxide, Silica, Methicone, Alumina |
| C | 3.0 | Glycerin |
| | 0.2 | Disodium EDTA |
| | 0.3 | Xanthan Gum |
| | 1.0 | Decyl Glucoside |
| | 2.0 | Panthenol, Propylene Glycol |
| | 56.3 | Aqua dem. |
| D | 1.0 | Aqueous solution with about 5% hydrophobin |
| | 1.0 | Tocopheryl Acetate |
| | 0.2 | Bisabolol |
| | q.s. | Perfume oil |
| | q.s. | Preservative |

### WS 5%:

| | % | Ingredients (INCI) |
|---|---|---|
| A | 3.5 | Ceteareth-6, Stearyl Alcohol |
| | 1.5 | Ceteareth-25 |
| | 7.5 | Ethylhexyl Methoxycinnamate |
| | 2.0 | Diethylamino Hydroxybenzoyl Hexyl Benzoate |
| | 2.0 | Cyclopentasiloxane, Cyclohexasiloxane |
| | 0.5 | Bees Wax |
| | 3.0 | Cetearyl Alcohol |
| | 10.0 | Caprylic/Capric Triglyceride |
| B | 5.0 | Titanium Dioxide, Silica, Methicone, Alumina |
| C | 3.0 | Glycerin |
| | 0.2 | Disodium EDTA |
| | 0.3 | Xanthan Gum |
| | 1.0 | Decyl Glucoside |
| | 2.0 | Panthenol, Propylene Glycol |
| | 52.3 | Aqua dem. |
| D | 5.0 | Aqueous solution with about 5% hydrophobin |
| | 1.0 | Tocopheryl Acetate |
| | 0.2 | Bisabolol |
| | q.s. | Perfume oil |
| | q.s. | Preservative |

Preparation: Heat phase A to about 80° C., stir in phase B and homogenize for 3 min. Likewise heat phase C to 80° C. and stir into the combined phases A and B with homogenization. Cool to about 40° C., stir in phase D and homogenize again.

### Example 25

### Use of the Hydrophobin in a Foot Balsam

### WS 1%:

| | % | Ingredients (INCI) |
|---|---|---|
| A | 2.0 | Ceteareth-6, Stearyl Alcohol |
| | 2.0 | Ceteareth-25 |
| | 5.0 | Cetearyl Ethylhexanoate |
| | 4.0 | Cetyl Alcohol |
| | 4.0 | Glyceryl Stearate |
| | 5.0 | Mineral Oil |
| | 0.2 | Menthol |
| | 0.5 | Camphor |
| B | 69.3 | Aqua dem. |
| | q.s. | Preservative |
| C | 1.0 | Bisabolol |
| | 1.0 | Tocopheryl Acetate |
| D | 1.0 | Aqueous solution with about 5% hydrophobin |
| | 5.0 | Witch Hazel Extract |

### WS 5%:

| | % | Ingredients (INCI) |
|---|---|---|
| A | 2.0 | Ceteareth-6, Stearyl Alcohol |
| | 2.0 | Ceteareth-25 |
| | 5.0 | Cetearyl Ethylhexanoate |
| | 4.0 | Cetyl Alcohol |
| | 4.0 | Glyceryl Stearate |
| | 5.0 | Mineral Oil |
| | 0.2 | Menthol |
| | 0.5 | Camphor |
| B | 65.3 | Aqua dem. |
| | q.s. | Preservative |
| C | 1.0 | Bisabolol |
| | 1.0 | Tocopheryl Acetate |
| D | 5.0 | Aqueous solution with about 5% hydrophobin |
| | 5.0 | Witch Hazel Extract |

Preparation: Heat the components of phases A and B separately from one another to about 80° C. Stir phase B into phase A with homogenization. Cool to about 40° C. with stirring, add phases C and D and briefly after-homogenize. Cool to room temperature with stirring.

### Example 26

### Use of the Hydrophobin in a W/O Emulsion with Bisabolol

### WS 1%:

| | % | Ingredients (INCI) |
|---|---|---|
| A | 6.0 | PEG-7 Hydrogenated Castor Oil |
| | 8.0 | Cetearyl Ethylhexanoate |
| | 5.0 | Isopropyl Myristate |
| | 15.0 | Mineral Oil |
| | 0.3 | Magnesium Stearate |
| | 0.3 | Aluminum Stearate |
| | 2.0 | PEG-45/Dodecyl Glycol Copolymer |
| B | 5.0 | Glycerin |
| | 0.7 | Magnesium Sulfate |
| | 55.6 | Aqua dem. |
| C | 1.0 | Aqueous solution with about 5% hydrophobin |
| | 0.5 | Tocopheryl Acetate |
| | 0.6 | Bisabolol |

### WS 5%:

| | % | Ingredients (INCI) |
|---|---|---|
| A | 6.0 | PEG-7 Hydrogenated Castor Oil |
| | 8.0 | Cetearyl Ethylhexanoate |
| | 5.0 | Isopropyl Myristate |
| | 15.0 | Mineral Oil |
| | 0.3 | Magnesium Stearate |
| | 0.3 | Aluminum Stearate |
| | 2.0 | PEG-45/Dodecyl Glycol Copolymer |
| B | 5.0 | Glycerin |
| | 0.7 | Magnesium Sulfate |
| | 51.6 | Aqua dem. |
| C | 5.0 | Aqueous solution with about 5% hydrophobin |
| | 0.5 | Tocopheryl Acetate |

Preparation: Heat phases A and B separately from one another to about 85° C. Stir phase B into phase A and homogenize. Cool to about 40° C. with stirring, add phase C and briefly homogenize again. Cool to room temperature with stirring.

Assignment of the sequence names to DNA and polypeptide sequences in the sequence protocol

| | |
|---|---|
| dewA DNA and polypeptide sequence | SEQ ID NO: 1 |
| dewA polypeptide sequence | SEQ ID NO: 2 |
| rodA DNA and polypeptide sequence | SEQ ID NO: 3 |
| rodA polypeptide sequence | SEQ ID NO: 4 |
| hypA DNA and polypeptide sequence | SEQ ID NO: 5 |
| hypA polypeptide sequence | SEQ ID NO: 6 |
| hypB DNA and polypeptide sequence | SEQ ID NO: 7 |
| hypB polypeptide sequence | SEQ ID NO: 8 |
| sc3 DNA and polypeptide sequence | SEQ ID NO: 9 |
| sc3 polypeptide sequence | SEQ ID NO: 10 |
| basf1 DNA and polypeptide sequence | SEQ ID NO: 11 |
| basf1 polypeptide sequence | SEQ ID NO: 12 |
| basf2 DNA and polypeptide sequence | SEQ ID NO: 13 |
| basf2 polypeptide sequence | SEQ ID NO: 14 |
| yaad DNA and polypeptide sequence | SEQ ID NO: 15 |
| yaad polypeptide sequence | SEQ ID NO: 16 |
| yaae DNA and polypeptide sequence | SEQ ID NO: 17 |
| yaae polypeptide sequence | SEQ ID NO: 18 |
| yaad-Xa-dewA-his DNA and polypeptide | SEQ ID NO: 19 |
| sequence | |
| yaad-Xa-dewA-his polypeptide sequence | SEQ ID NO: 20 |
| yaad-Xa-rodA-his DNA and polypeptide | SEQ ID NO: 21 |
| sequence | |
| yaad-Xa-rodA-his polypeptide sequence | SEQ ID NO: 22 |
| yaad-Xa-basf1-his DNA and polypeptide | SEQ ID NO: 23 |
| sequence | |
| yaad-Xa-basf1-his polypeptide sequence | SEQ ID NO: 24 |

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. A cosmetic composition in a cosmetically compatible medium for the treatment of at least one of keratin-containing materials, mucosa, and teeth comprising at least one hydrophobin polypeptide sequence (I)
(I)
Xₙ-C¹-X₁₋₅₀-C²-X₀₋₅-C³-Xₚ-C⁴-X₁₋₁₀₀-C⁵-X₁₋₅₀-C⁶-
X₀₋₅-C⁷-X₁₋₅₀-C⁸-Xₘ
wherein X independently is any of the 20 naturally occurring amino acids, the numerical subscripts of X indicate the number of amino acids of each X, subscripts n and m of X indicate numbers between 0 and 500, C is cysteine, alanine, serine, glycine, methionine or threonine wherein at least four residues designated as C are cysteine, at least one of the peptide sequences abbreviated Xₙ or Xₘ or Xₚ is a peptide sequence of at least 20 amino acids that is not naturally linked to a hydrophobin, and wherein the polypeptide changes the contact angle by at least 20° after coating a glass surface.

15. The cosmetic composition of claim 14 wherein the hydrophobin polypeptide sequence (I) has binding affinity to one or more of human or animal hair keratin, nail keratin, skin keratin, mucosa or teeth.

16. The cosmetic composition of claim 14 wherein at least one of Xₙ or Xₘ or Xₚ is a human keratin-binding domain.

17. The cosmetic composition of claim 14 comprising 0.000001 to 10% by weight of the hydrophobin polypeptide sequence (I).

18. The cosmetic composition of claim 14 comprising at least one cosmetic active substance in addition to the hydrophobin polypeptide sequence (I).

19. The cosmetic composition of claim 18 wherein the cosmetic active substance is selected from the group consisting of natural or synthetic polymers, pigments, humectants, oils, waxes, enzymes, minerals, vitamins, sunscreens, dyes, fragrances, antioxidants, and preservatives.

20. A conjugate of hydrophobin and at least one active substance and at least one effect substance comprising a hydrophobin covalently linked to an effector molecule.

21. The conjugate of claim 20 wherein the effector molecule is selected from the group consisting of dyes, antioxidants, UV filters, vitamins, fungicides, insecticides, and biocides.

22. The conjugate of claim 20 wherein the hydrophobin is the hydrophobin polypeptide sequence (I) of claim 1.

23. A cosmetic composition for the treatment of keratin-containing materials comprising at least one of the conjugate of claim 20.


SEQUENCE LISTING (35 sequences)

SEQ ID NO: 1 - DNA, 405 bp, Unknown
  CDS (1)..(405): basf-dewA
atgcgcttcatcgtctctctcctcgccttcactgccgcggccaccgcgaccgccctcccggcctctgccgcaaagaacgcgaagctggccacctcggcggccttcgccaagcaggctgaaggcaccacctgcaatgtcggctcgatcgcttgctgcaactcccccgctgagaccaacaacgacagtctgttgagcggtctgctcggtgctggccttctcaacgggctctcgggcaacactggcagcgcctgcgccaaggcgagcttgattgaccagctgggtctgctcgctctcgtcgaccacactgaggaaggccccgtctgcaagaacatcgtcgcttgctgccctgagggaaccaccaactgtgttgccgtcgacaacgctggcgctggtaccaaggctgag

SEQ ID NO: 2 - protein, 135 aa, Unknown
  basf-dewA
MRFIVSLLAFTAAATATALPASAAKNAKLATSAAFAKQAEGTTCNVGSIACCNSPAETNNDSLLSGLLGAGLLNGLSGNTGSACAKASLIDQLGLLALVDHTEEGPVCKNIVACCPEGTTNCVAVDNAGAGTKAE

SEQ ID NO: 3 - DNA, 471 bp, Unknown
  CDS (1)..(471): basf-rodA
atgaagttctccattgctgccgctgtcgttgctttcgccgcctccgtcgcggccctccctcctgcccatgattcccagttcgctggcaatggtgttggcaacaagggcaacagcaacgtcaagttccctgtccccgaaaacgtgaccgtcaagcaggcctccgacaagtgcggtgaccaggcccagctctcttgctgcaacaaggccacgtacgccggtgacaccacaaccgttgatgagggtcttctgtctggtgccctcagcggcctcatcggcgccgggtctggtgccgaaggtcttggtctcttcgatcagtgctccaagcttgatgttgctgtcctcattggcatccaagatcttgtcaaccagaagtgcaagcaaaacattgcctgctgccagaactccccctccagcgcggatggcaaccttattggtgtcggtctcccttgcgttgcccttggctccatcctc

SEQ ID NO: 4 - protein, 157 aa, Unknown
  basf-rodA
MKFSIAAAVVAFAASVAALPPAHDSQFAGNGVGNKGNSNVKFPVPENVTVKQASDKCGDQAQLSCCNKATYAGDTTTVDEGLLSGALSGLIGAGSGAEGLGLFDQCSKLDVAVLIGIQDLVNQKCKQNIACCQNSPSSADGNLIGVGLPCVALGSIL

SEQ ID NO: 5 - DNA, 336 bp, Unknown
  CDS (1)..(336): basf-HypA
atgatctctcgcgtccttgtcgctgctctcgtcgctctccccgctcttgttactgcaactcctgctcccggaaagcctaaagccagcagtcagtgcgacgtcggtgaaatccattgctgtgacactcagcagactcccgaccacaccagcgccgccgcgtctggtttgcttggtgttcccatcaaccttggtgctttcctcggtttcgactgtacccccatttccgtccttggcgtcggtggcaacaactgtgctgctcagcctgtctgctgcacaggaaatcaattcaccgcattgattaacgctcttgactgctctcctgtcaatgtcaacctc

SEQ ID NO: 6 - protein, 112 aa, Unknown
  basf-HypA
MISRVLVAALVALPALVTATPAPGKPKASSQCDVGEIHCCDTQQTPDHTSAAASGLLGVPINLGAFLGFDCTPISVLGVGGNNCAAQPVCCTGNQFTALINALDCSPVNVNL

SEQ ID NO: 7 - DNA, 357 bp, Unknown
  CDS (1)..(357): basf-HypB
atggtcagcacgttcatcactgtcgcaaagacccttctcgtcgcgctcctcttcgtcaatatcaatatcgtcgttggtactgcaactaccggcaagcattgtagcaccggtcctatcgagtgctgcaagcaggtcatggattctaagagccctcaggctacggagcttcttacgaagaatggccttggcctgggtgtccttgctggcgtgaagggtcttgttggcgcgaattgcagccctatcacggcaattggtattggctccggcagccaatgctctggccagaccgtttgctgccagaataataatttcaacggtgttgtcgctattggttgcactcccattaatgccaatgtg

SEQ ID NO: 8 - protein, 119 aa, Unknown
  basf-HypB
MVSTFITVAKTLLVALLFVNINIVVGTATTGKHCSTGPIECCKQVMDSKSPQATELLTKNGLGLGVLAGVKGLVGANCSPITAIGIGSGSQCSGQTVCCQNNNFNGVVAIGCTPINANV

SEQ ID NO: 9 - DNA, 408 bp, Unknown
  CDS (1)..(408): basf-sc3
atgttcgcccgtctccccgtcgtgttcctctacgccttcgtcgcgttcggcgccctcgtcgctgccctcccaggtggccacccgggcacgaccacgccgccggttacgacgacggtgacggtgaccacgccgccctcgacgacgaccatcgccgccggtggcacgtgtactacggggtcgctctcttgctgcaaccaggttcaatcggcgagcagcagccctgttaccgccctcctcggcctgctcggcattgtcctcagcgacctcaacgttctcgttggcatcagctgctctcccctcactgtcatcggtgtcggaggcagcggctgttcggcgcagaccgtctgctgcgaaaacacccaattcaacgggctgatcaacatcggttgcacccccatcaacatcctc

SEQ ID NO: 10 - protein, 136 aa, basf-sc3
  basf-sc3
MFARLPVVFLYAFVAFGALVAALPGGHPGTTTPPVTTTVTVTTPPSTTTIAAGGTCTTGSLSCCNQVQSASSSPVTALLGLLGIVLSDLNVLVGISCSPLTVIGVGGSGCSAQTVCCENTQFNGLINIGCTPINIL

SEQ ID NO: 11 - DNA, 483 bp, Unknown
  CDS (1)..(483): basf-BASF1
atgaagttctccgtctccgccgccgtcctcgccttcgccgcctccgtcgccgccctccctcagcacgactccgccgccggcaacggcaacggcgtcggcaacaagttccctgtccctgacgacgtcaccgtcaagcaggccaccgacaagtgcggcgaccaggcccagctctcctgctgcaacaaggccacctacgccggcgacgtcctcaccgacatcgacgagggcatcctcgccggcctcctcaagaacctcatcggcggcggctccggctccgagggcctcggcctcttcgaccagtgcgtcaagctcgacctccagatctccgtcatcggcatccctatccaggacctcctcaaccaggtcaacaagcagtgcaagcagaacatcgcctgctgccagaactccccttccgacgccaccggctccctcgtcaacctcggcctcggcaacccttgcatccctgtctccctcctccatatg

SEQ ID NO: 12 - protein, 161 aa, Unknown
  basf-BASF1
MKFSVSAAVLAFAASVAALPQHDSAAGNGNGVGNKFPVPDDVTVKQATDKCGDQAQLSCCNKATYAGDVLTDIDEGILAGLLKNLIGGGSGSEGLGLFDQCVKLDLQISVIGIPIQDLLNQVNKQCKQNIACCQNSPSDATGSLVNLGLGNPCIPVSLLHM

SEQ ID NO: 13 - DNA, 465 bp, Unknown
  CDS (1)..(465): basf-BASF2
atgaagttctccgtctccgccgccgtcctcgccttcgccgcctccgtcgccgccctccctcagcacgactccgccgccggcaacggcaacggcgtcggcaacaagttccctgtccctgacgacgtcaccgtcaagcaggccaccgacaagtgcggcgaccaggcccagctctcctgctgcaacaaggccacctacgccggcgacgtcaccgacatcgacgagggcatcctcgccggcctcctcaagaacctcatcggcggcggctccggctccgagggcctcggcctcttcgaccagtgcgtcaagctcgacctccagatctccgtcatcggcatccctatccaggacctcctcaaccagcagtgcaagcagaacatcgcctgctgccagaactccccttccgacgccaccggctccctcgtcaacctcggcaacccttgcatccctgtctccctcctccatatg

SEQ ID NO: 14 - protein, 155 aa, Unknown
  basf-BASF2
MKFSVSAAVLAFAASVAALPQHDSAAGNGNGVGNKFPVPDDVTVKQATDKCGDQAQLSCCNKATYAGDVTDIDEGILAGLLKNLIGGGSGSEGLGLFDQCVKLDLQISVIGIPIQDLLNQQCKQNIACCQNSPSDATGSLVNLGNPCIPVSLLHM

SEQ ID NO: 15 - DNA, 882 bp, Unknown
  CDS (1)..(882): basf-yaad
atggctcaaacaggtactgaacgtgtaaaacgcggaatggcagaaatgcaaaaaggcggcgtcatcatggacgtcatcaatgcggaacaagcgaaaatcgctgaagaagctggagctgtcgctgtaatggcgctagaacgtgtgccagcagatattcgcgcggctggaggagttgcccgtatggctgaccctacaatcgtggaagaagtaatgaatgcagtatctatcccggtaatggcaaaagcgcgtatcggacatattgttgaagcgcgtgtgcttgaagctatgggtgttgactatattgatgaaagtgaagttctgacgccggctgacgaagaatttcatttaaataaaaatgaatacacagttccttttgtctgtggctgccgtgatcttggtgaagcaacacgccgtattgcggaaggtgcttctatgcttcgcacaaaaggtgagcctggaacaggtaatattgttgaggctgttcgccatatgcgtaaagttaacgctcaagtgcgcaaagtagttgcgatgagtgaggatgagctaatgacagaagcgaaaaacctaggtgctccttacgagcttcttcttcaaattaaaaaagacggcaagcttcctgtcgttaactttgccgctggcggcgtagcaactccagctgatgctgctctcatgatgcagcttggtgctgacggagtatttgttggttctggtatttttaaatcagacaaccctgctaaatttgcgaaagcaattgtggaagcaacaactcactttactgattacaaattaatcgctgagttgtcaaaagagcttggtactgcaatgaaagggattgaaatctcaaacttacttccagaacagcgtatgcaagaacgcggctgg

SEQ ID NO: 16 - protein, 294 aa, basf-yaad
  basf-yaad
MAQTGTERVKRGMAEMQKGGVIMDVINAEQAKIAEEAGAVAVMALERVPADIRAAGGVARMADPTIVEEVMNAVSIPVMAKARIGHIVEARVLEAMGVDYIDESEVLTPADEEFHLNKNEYTVPFVCGCRDLGEATRRIAEGASMLRTKGEPGTGNIVEAVRHMRKVNAQVRKVVAMSEDELMTEAKNLGAPYELLLQIKKDGKLPVVNFAAGGVATPADAALMMQLGADGVFVGSGIFKSDNPAKFAKAIVEATTHFTDYKLIAELSKELGTAMKGIEISNLLPEQRMQERGW

SEQ ID NO: 17 - DNA, 591 bp, Unknown
  CDS (1)..(591): basf-yaae
atgggattaacaataggtgtactaggacttcaaggagcagttagagagcacatccatgcgattgaagcatgcggcgcggctggtcttgtcgtaaaacgtccggagcagctgaacgaagttgacgggttgattttgccgggcggtgagagcacgacgatgcgccgtttgatcgatacgtatcaattcatggagccgcttcgtgaattcgctgctcagggcaaaccgatgtttggaacatgtgccggattaattatattagcaaaagaaattgccggttcagataatcctcatttaggtcttctgaatgtggttgtagaacgtaattcatttggccggcaggttgacagctttgaagctgatttaacaattaaaggcttggacgagccttttactggggtattcatccgtgctccgcatattttagaagctggtgaaaatgttgaagttctatcggagcataatggtcgtattgtagccgcgaaacaggggcaattccttggctgctcattccatccggagctgacagaagatcaccgagtgacgcagctgtttgttgaaatggttgaggaatataagcaaaaggcacttgta

SEQ ID NO: 18 - protein, 197 aa, Unknown
  basf-yaae
MGLTIGVLGLQGAVREHIHAIEACGAAGLVVKRPEQLNEVDGLILPGGESTTMRRLIDTYQFMEPLREFAAQGKPMFGTCAGLIILAKEIAGSDNPHLGLLNVVVERNSFGRQVDSFEADLTIKGLDEPFTGVFIRAPHILEAGENVEVLSEHNGRIVAAKQGQFLGCSFHPELTEDHRVTQLFVEMVEEYKQKALV

SEQ ID NO: 19 - DNA, 1329 bp, Unknown
  CDS (1)..(1329): basf-yaad-Xa-dewA-his
atggctcaaacaggtactgaacgtgtaaaacgcggaatggcagaaatgcaaaaaggcggcgtcatcatggacgtcatcaatgcggaacaagcgaaaatcgctgaagaagctggagctgtcgctgtaatggcgctagaacgtgtgccagcagatattcgcgcggctggaggagttgcccgtatggctgaccctacaatcgtggaagaagtaatgaatgcagtatctatcccggtaatggcaaaagcgcgtatcggacatattgttgaagcgcgtgtgcttgaagctatgggtgttgactatattgatgaaagtgaagttctgacgccggctgacgaagaatttcatttaaataaaaatgaatacacagttccttttgtctgtggctgccgtgatcttggtgaagcaacacgccgtattgcggaaggtgcttctatgcttcgcacaaaaggtgagcctggaacaggtaatattgttgaggctgttcgccatatgcgtaaagttaacgctcaagtgcgcaaagtagttgcgatgagtgaggatgagctaatgacagaagcgaaaaacctaggtgctccttacgagcttcttcttcaaattaaaaaagacggcaagcttcctgtcgttaactttgccgctggcggcgtagcaactccagctgatgctgctctcatgatgcagcttggtgctgacggagtatttgttggttctggtatttttaaatcagacaaccctgctaaatttgcgaaagcaattgtggaagcaacaactcactttactgattacaaattaatcgctgagttgtcaaaagagcttggtactgcaatgaaagggattgaaatctcaaacttacttccagaacagcgtatgcaagaacgcggctggagatccattgaaggccgcatgcgcttcatcgtctctctcctcgccttcactgccgcggccaccgcgaccgccctcccggcctctgccgcaaagaacgcgaagctggccacctcggcggccttcgccaagcaggctgaaggcaccacctgcaatgtcggctcgatcgcttgctgcaactcccccgctgagaccaacaacgacagtctgttgagcggtctgctcggtgctggccttctcaacgggctctcgggcaacactggcagcgcctgcgccaaggcgagcttgattgaccagctgggtctgctcgctctcgtcgaccacactgaggaaggccccgtctgcaagaacatcgtcgcttgctgccctgagggaaccaccaactgtgttgccgtcgacaacgctggcgctggtaccaaggctgagggatctcatcaccatcaccatcac

SEQ ID NO: 20 - protein, 443 aa, Unknown
  basf-yaad-Xa-dewA-his
MAQTGTERVKRGMAEMQKGGVIMDVINAEQAKIAEEAGAVAVMALERVPADIRAAGGVARMADPTIVEEVMNAVSIPVMAKARIGHIVEARVLEAMGVDYIDESEVLTPADEEFHLNKNEYTVPFVCGCRDLGEATRRIAEGASMLRTKGEPGTGNIVEAVRHMRKVNAQVRKVVAMSEDELMTEAKNLGAPYELLLQIKKDGKLPVVNFAAGGVATPADAALMMQLGADGVFVGSGIFKSDNPAKFAKAIVEATTHFTDYKLIAELSKELGTAMKGIEISNLLPEQRMQERGWRSIEGRMRFIVSLLAFTAAATATALPASAAKNAKLATSAAFAKQAEGTTCNVGSIACCNSPAETNNDSLLSGLLGAGLLNGLSGNTGSACAKASLIDQLGLLALVDHTEEGPVCKNIVACCPEGTTNCVAVDNAGAGTKAEGSHHHHHH

SEQ ID NO: 21 - DNA, 1395 bp, Unknown
  CDS (1)..(1395): basf-yaad-Xa-rodA-his
atggctcaaacaggtactgaacgtgtaaaacgcggaatggcagaaatgcaaaaaggcggcgtcatcatggacgtcatcaatgcggaacaagcgaaaatcgctgaagaagctggagctgtcgctgtaatggcgctagaacgtgtgccagcagatattcgcgcggctggaggagttgcccgtatggctgaccctacaatcgtggaagaagtaatgaatgcagtatctatcccggtaatggcaaaagcgcgtatcggacatattgttgaagcgcgtgtgcttgaagctatgggtgttgactatattgatgaaagtgaagttctgacgccggctgacgaagaatttcatttaaataaaaatgaatacacagttccttttgtctgtggctgccgtgatcttggtgaagcaacacgccgtattgcggaaggtgcttctatgcttcgcacaaaaggtgagcctggaacaggtaatattgttgaggctgttcgccatatgcgtaaagttaacgctcaagtgcgcaaagtagttgcgatgagtgaggatgagctaatgacagaagcgaaaaacctaggtgctccttacgagcttcttcttcaaattaaaaaagacggcaagcttcctgtcgttaactttgccgctggcggcgtagcaactccagctgatgctgctctcatgatgcagcttggtgctgacggagtatttgttggttctggtatttttaaatcagacaaccctgctaaatttgcgaaagcaattgtggaagcaacaactcactttactgattacaaattaatcgctgagttgtcaaaagagcttggtactgcaatgaaagggattgaaatctcaaacttacttccagaacagcgtatgcaagaacgcggctggagatctattgaaggccgcatgaagttctccattgctgccgctgtcgttgctttcgccgcctccgtcgcggccctccctcctgcccatgattcccagttcgctggcaatggtgttggcaacaagggcaacagcaacgtcaagttccctgtccccgaaaacgtgaccgtcaagcaggcctccgacaagtgcggtgaccaggcccagctctcttgctgcaacaaggccacgtacgccggtgacaccacaaccgttgatgagggtcttctgtctggtgccctcagcggcctcatcggcgccgggtctggtgccgaaggtcttggtctcttcgatcagtgctccaagcttgatgttgctgtcctcattggcatccaagatcttgtcaaccagaagtgcaagcaaaacattgcctgctgccagaactccccctccagcgcggatggcaaccttattggtgtcggtctcccttgcgttgcccttggctccatcctcggatctcatcaccatcaccatcac

SEQ ID NO: 22 - protein, 465 aa, Unknown
  basf-yaad-Xa-rodA-his
MAQTGTERVKRGMAEMQKGGVIMDVINAEQAKIAEEAGAVAVMALERVPADIRAAGGVARMADPTIVEEVMNAVSIPVMAKARIGHIVEARVLEAMGVDYIDESEVLTPADEEFHLNKNEYTVPFVCGCRDLGEATRRIAEGASMLRTKGEPGTGNIVEAVRHMRKVNAQVRKVVAMSEDELMTEAKNLGAPYELLLQIKKDGKLPVVNFAAGGVATPADAALMMQLGADGVFVGSGIFKSDNPAKFAKAIVEATTHFTDYKLIAELSKELGTAMKGIEISNLLPEQRMQERGWRSIEGRMKFSIAAAVVAFAASVAALPPAHDSQFAGNGVGNKGNSNVKFPVPENVTVKQASDKCGDQAQLSCCNKATYAGDTTTVDEGLLSGALSGLIGAGSGAEGLGLFDQCSKLDVAVLIGIQDLVNQKCKQNIACCQNSPSSADGNLIGVGLPCVALGSILGSHHHHHH

SEQ ID NO: 23 - DNA, 1407 bp, Unknown
  CDS (1)..(1407): basf-yaad-Xa-BASF1-his
atggctcaaacaggtactgaacgtgtaaaacgcggaatggcagaaatgcaaaaaggcggcgtcatcatggacgtcatcaatgcggaacaagcgaaaatcgctgaagaagctggagctgtcgctgtaatggcgctagaacgtgtgccagcagatattcgcgcggctggaggagttgcccgtatggctgaccctacaatcgtggaagaagtaatgaatgcagtatctatcccggtaatggcaaaagcgcgtatcggacatattgttgaagcgcgtgtgcttgaagctatgggtgttgactatattgatgaaagtgaagttctgacgccggctgacgaagaatttcatttaaataaaaatgaatacacagttccttttgtctgtggctgccgtgatcttggtgaagcaacacgccgtattgcggaaggtgcttctatgcttcgcacaaaaggtgagcctggaacaggtaatattgttgaggctgttcgccatatgcgtaaagttaacgctcaagtgcgcaaagtagttgcgatgagtgaggatgagctaatgacagaagcgaaaaacctaggtgctccttacgagcttcttcttcaaattaaaaaagacggcaagcttcctgtcgttaactttgccgctggcggcgtagcaactccagctgatgctgctctcatgatgcagcttggtgctgacggagtatttgttggttctggtatttttaaatcagacaaccctgctaaatttgcgaaagcaattgtggaagcaacaactcactttactgattacaaattaatcgctgagttgtcaaaagagcttggtactgcaatgaaagggattgaaatctcaaacttacttccagaacagcgtatgcaagaacgcggctggagatctattgaaggccgcatgaagttctccgtctccgccgccgtcctcgccttcgccgcctccgtcgccgccctccctcagcacgactccgccgccggcaacggcaacggcgtcggcaacaagttccctgtccctgacgacgtcaccgtcaagcaggccaccgacaagtgcggcgaccaggcccagctctcctgctgcaacaaggccacctacgccggcgacgtcctcaccgacatcgacgagggcatcctcgccggcctcctcaagaacctcatcggcggcggctccggctccgagggcctcggcctcttcgaccagtgcgtcaagctcgacctccagatctccgtcatcggcatccctatccaggacctcctcaaccaggtcaacaagcagtgcaagcagaacatcgcctgctgccagaactccccttccgacgccaccggctccctcgtcaacctcggcctcggcaacccttgcatccctgtctccctcctccatatgggatctcatcaccatcaccatcac

SEQ ID NO: 24 - protein, 469 aa, Unknown
  basf-yaad-Xa-BASF1-his
MAQTGTERVKRGMAEMQKGGVIMDVINAEQAKIAEEAGAVAVMALERVPADIRAAGGVARMADPTIVEEVMNAVSIPVMAKARIGHIVEARVLEAMGVDYIDESEVLTPADEEFHLNKNEYTVPFVCGCRDLGEATRRIAEGASMLRTKGEPGTGNIVEAVRHMRKVNAQVRKVVAMSEDELMTEAKNLGAPYELLLQIKKDGKLPVVNFAAGGVATPADAALMMQLGADGVFVGSGIFKSDNPAKFAKAIVEATTHFTDYKLIAELSKELGTAMKGIEISNLLPEQRMQERGWRSIEGRMKFSVSAAVLAFAASVAALPQHDSAAGNGNGVGNKFPVPDDVTVKQATDKCGDQAQLSCCNKATYAGDVLTDIDEGILAGLLKNLIGGGSGSEGLGLFDQCVKLDLQISVIGIPIQDLLNQVNKQCKQNIACCQNSPSDATGSLVNLGLGNPCIPVSLLHMGSHHHHHH

SEQ ID NO: 25 - DNA, 28 bp, Unknown
  Hal570 oligonucleotide
gcgcgcccatggctcaaacaggtactga

SEQ ID NO: 26 - DNA, 28 bp, Unknown
  Hal571 oligonucleotide
gcagatctccagccgcgttcttgcatac

SEQ ID NO: 27 - DNA, 30 bp, Unknown
  Hal572 oligonucleotide
ggccatgggattaacaataggtgtactagg

SEQ ID NO: 28 - DNA, 33 bp, Unknown
  Hal573 oligonucleotide
gcagatcttacaagtgccttttgcttatattcc

SEQ ID NO: 29 - DNA, 38 bp, Unknown
  KaM416 oligonucleotide
gcagcccatcagggatccctcagccttggtaccagcgc

SEQ ID NO: 30 - DNA, 50 bp, Unknown
  KaM417 oligonucleotide
cccgtagctagtggatccattgaaggccgcatgaagttctccgtctccgc

SEQ ID NO: 31 - DNA, 45 bp, Unknown
  KaM434 oligonucleotide
gctaagcggatccattgaaggccgcatgaagttctccattgctgc

SEQ ID NO: 32 - DNA, 30 bp, Unknown
  KaM435 oligonucleotide
ccaatggggatccgaggatggagccaaggg

SEQ ID NO: 33 - DNA, 38 bp, Unknown
  KaM418 oligonucleotide
ctgccattcaggggatcccatatggaggagggagacag

SEQ ID NO: 34 - DNA, 32 bp, Unknown
  KaM464 oligonucleotide
cgttaaggatccgaggatgttgatgggggtgc

SEQ ID NO: 35 - DNA, 35 bp, Unknown
  KaM465 oligonucleotide
gctaacagatctatgttcgcccgtctccccgtcgt